(19) United States
(12) Patent Application Publication
(10) Pub. No.: US 2005/0054708 A1
(43) Pub. Date: Mar. 10, 2005

(54) Combinations of drugs for the treatment of neoplasms

(76) Inventors: Matthew James Nichols, Boston, MA (US); Margaret S. Lee, Middleton, MA (US); Curtis Keith, Boston, MA (US); Yanzhen Zhang, Sudbury, MA (US); Debra A. Gaw, Reading, MA (US)

Correspondence Address: CLARK & ELBING LLP, 101 FEDERAL STREET, BOSTON, MA, 02110, US

(21) Appl. No.: 10/895,561

(22) Filed: Jul. 21, 2004

Related U.S. Application Data
(60) Provisional application No. 60/490,759, filed on Jul. 28, 2003.

(51) Int. Cl. A61K 31/40; A61K 31/381; A61K 31/34; A61K 31/155

(52) U.S. Cl. 514/408; 514/461; 514/438; 514/485; 514/632

(57) ABSTRACT

The invention features a method for treating a patient having a cancer or other neoplasm by administering to the patient pentamidine or a pentamidine analog and an antiproliferative agent simultaneously or within 14 days of each other in amounts sufficient to treat the patient.

### CROSS-REFERENCE TO RELATED APPLICATIONS

This application claims benefit from U.S. Application No. 60/490,759, filed Jul. 28, 2003, which is hereby incorporated by reference.

### BACKGROUND OF THE INVENTION

The invention relates to the treatment of neoplasms such as cancer.

Cancer is a disease marked by the uncontrolled growth of abnormal cells. Cancer cells have overcome the barriers imposed on normal cells, which have a finite lifespan, to grow indefinitely. As the growth of cancer cells continue, genetic alterations may persist until the cancerous cell has manifested itself to pursue a more aggressive growth phenotype. If left untreated, metastasis, the spread of cancer cells to distant areas of the body by way of the lymph system or bloodstream, may ensue, destroying healthy tissue.

According to a recent American Cancer Society study, approximately 1,268,000 new cancer cases were expected to be diagnosed in the United States in the year 2001 alone. Lung cancer is the most common cancer-related cause of death among men and women, accounting for over 28% of all cancer-related deaths. It is the second most commonly occurring cancer among men and women; it has been estimated that there were more than 169,000 new cases of lung cancer in the U.S. in the year 2001 and accounting for 13% of all new cancer diagnoses. While the rate of lung cancer cases is declining among men in the U.S., it continues to increase among women. According to the American Cancer Society, an estimated 157,400 Americans were expected to die due to lung cancer in 2001.

Cancers that begin in the lungs are divided into two major types, non-small cell lung cancer and small cell lung cancer, depending on how the cells appear under a microscope. Non-small cell lung cancer (squamous cell carcinoma, adenocarcinoma, and large cell carcinoma) generally spreads to other organs more slowly than does small cell lung cancer. Small cell lung cancer is the less common type, accounting for about 20% of all lung cancer.

Other cancers include brain cancer, breast cancer, cervical cancer, colon cancer, gastric cancer, kidney cancer, leukemia, liver cancer, lymphoma, ovarian cancer, pancreatic cancer, prostate cancer, rectal cancer, sarcoma, skin cancer, testicular cancer, and uterine cancer. These cancers, like lung cancer, are sometimes treated with chemotherapy.

Despite the availability of numerous chemotherapeutic agents, there is still a need for treatment regimens for certain cancers, as well as a general desire for safer, more efficacious chemotherapy regimens.

### SUMMARY OF THE INVENTION

The present invention features the combination of pentamidine, or a pentamidine analog or metabolite, with an antiproliferative agent for the treatment of a neoplasm.

Accordingly, in a first aspect, the invention features a method for treating a patient having a cancer or other neoplasm, or inhibiting the development of a neoplasm in a patient who is at risk for developing a neoplasm, by administering to the patient:
(a) a compound having the formula (I): 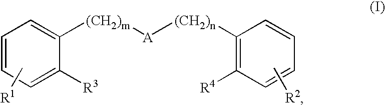
or a pharmaceutically acceptable salt thereof,
wherein A is 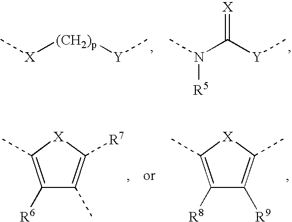
each of X and Y is, independently, O, NR¹⁰, or S,
each of R⁵ and R¹⁰ is, independently, H or C₁-C₆ alkyl,
each of R⁶, R⁷, R⁸, and R⁹ is, independently, H, C₁-C₆ alkyl, halogen, C₁-C₆
alkyloxy, C₆-C₁₈ aryloxy, or C₆-C₁₈ aryl-C₁-C₆ alkyloxy,
p is an integer between 2 and 6, inclusive,
each of m and n is, independently, an integer between 0 and 2, inclusive,
each of R¹ and R² is 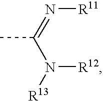
wherein R¹² is H, C₁-C₆ alkyl, C₁-C₈ cycloalkyl, C₁-C₆ alkyloxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent 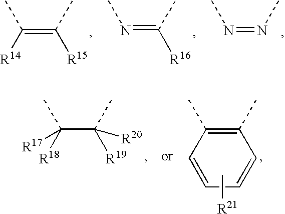
wherein each of R¹⁴, R¹⁵, and R¹⁶ is, independently, H, C₁-C₆ alkyl, halogen, or trifluoromethyl, each of R¹⁷, R¹⁸, R¹⁹, and R²⁰ is, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, or C₆-C₁₈ aryl,
each of R³ and R⁴ is, independently, H, Cl, Br, OH, OCH₃, OCF₃, NO₂, and NH₂, or R³ and R⁴ together form a single bond; and
b) one or more Group A antiproliferative agents.

By “Group A antiproliferative agent” is meant any antiproliferative agent that is not a Group B antiproliferative agent. Examples of Group A agents are those listed in Table 1. Group A antiproliferative agents of the invention also include those alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists and antagonists, endothelin A receptor antagonists, retinoic acid receptor agonists, immunomodulators, hormonal and antihormonal agents, photodynamic agents, and tyrosine kinase inhibitors that are not Group B antiproliferative agents, as defined herein (see Table 2).

TABLE 1
(Group A)

| | | |
|---|---|---|
| Alkylating agents | busulfan | procarbazine |
| | ifosfamide | altretamine |
| | hexamethylmelamine | estramustine phosphate |
| | thiotepa | mechlorethamine |
| | dacarbazine | streptozocin |
| | lomustine | temozolomide |
| | cyclophosphamide | semustine |
| | chlorambucil | |
| Platinum agents | spiroplatin | lobaplatin (Aeterna) |
| | tetraplatin | satraplatin (Johnson Matthey) |
| | ormaplatin | BBR-3464 (Hoffmann-La Roche) |
| | iproplatin | SM-11355 (Sumitomo) |
| | ZD-0473 (AnorMED) | AP-5280 (Access) |
| | oxaliplatin | |
| | carboplatin | |
| Antimetabolites | azacytidine | trimetrexate |
| | floxuridine | deoxycoformycin |
| | 2-chlorodeoxyadenosine | pentostatin |
| | 6-mercaptopurine | hydroxyurea |
| | 6-thioguanine | decitabine (SuperGen) |
| | cytarabine | clofarabine (Bioenvision) |
| | 2-fluorodeoxy cytidine | irofulven (MGI Pharma) |
| | methotrexate | DMDC (Hoffmann-La Roche) |
| | tomudex | ethynylcytidine (Taiho) |
| | fludarabine | gemcitabine |
| | raltitrexed | capecitabine |
| Topoisomerase | Amsacrine | exatecan mesylate (Daiichi) |
| inhibitors | epirubicin | quinamed (ChemGenex) |
| | etoposide | gimatecan (Sigma-Tau) |
| | teniposide or mitoxantrone | diflomotecan (Beaufour-Ipsen) |
| | 7-ethyl-10-hydroxy-camptothecin | TAS-103 (Taiho) |
| | dexrazoxanet (TopoTarget) | elsamitrucin (Spectrum) |
| | pixantrone (Novuspharma) | J-107088 (Merck & Co) |
| | rebeccamycin analogue (Exelixis) | BNP-1350 (BioNumerik) |
| | BBR-3576 (Novuspharma) | CKD-602 (Chong Kun Dang) |
| | rubitecan (SuperGen) | KW-2170 (Kyowa Hakko) |
| | irinotecan (CPT-11) | |
| | topotecan | |
| Antitumor | dactinomycin (actinomycin D) | azonafide |
| antibiotics | valrubicin | anthrapyrazole |
| | daunorubicin (daunomycin) | oxantrazole |
| | therarubicin | losoxantrone |
| | idarubicin | bleomycinic acid |
| | rubidazone | MEN-10755 (Menarini) |
| | plicamycinp | GPX-100 (Gem Pharmaceuticals) |
| | porfiromycin | epirubicin |
| | mitoxantrone (novantrone) | |
| | amonafide | |
| Antimitotic | colchicine | E7010 (Abbott) |
| agents | vinblastine | PG-TXL (Cell Therapeutics) |
| | vindesine | IDN 5109 (Bayer) |
| | dolastatin 10 (NCI) | A 105972 (Abbott) |
| | rhizoxin (Fujisawa) | A 204197 (Abbott) |
| | mivobulin (Warner-Lambert) | LU 223651 (BASF) |
| | cemadotin (BASF) | D 24851 (ASTAMedica) |
| | RPR 109881A (Aventis) | ER-86526 (Eisai) |
| | TXD 258 (Aventis) | combretastatin A4 (BMS) |
| | epothilone B (Novartis) | isohomohalichondrin-B (PharmaMar) |
| | T 900607 (Tularik) | ZD 6126 (AstraZeneca) |
| | T 138067 (Tularik) | AZ10992 (Asahi) |
| | cryptophycin 52 (Eli Lilly) | IDN-5109 (Indena) |
| | vinflunine (Fabre) | AVLB (Prescient NeuroPharma) |
| | auristatin PE (Teikoku Hormone) | azaepothilone B (BMS) |
| | BMS 247550 (BMS) | BNP-7787 (BioNumerik) |
| | BMS 184476 (BMS) | CA-4 prodrug (OXiGENE) |
| | BMS 188797 (BMS) | dolastatin-10 (NIH) |
| | taxoprexin (Protarga) | CA-4 (OXiGENE) |
| | SB 408075 (GlaxoSmithKline) | docetaxel |
| | Vinorelbine | vincristine |
| Aromatase | aminoglutethimide | YM-511 (Yamanouchi) |
| inhibitors | atamestane (BioMedicines) | formestane |
| | letrozole | exemestane |
| | anastrazole | |
| Thymidylate | pemetrexed (Eli Lilly) | nolatrexed (Eximias) |
| synthase inhibitors | ZD-9331 (BTG) | CoFactor ™ (BioKeys) |
| DNA antagonists | trabectedin (PharmaMar) | edotreotide (Novartis) |
| | glufosfamide (Baxter International) | mafosfamide (Baxter International) |
| | albumin + 32P (Isotope Solutions) | apaziquone (Spectrum Pharmaceuticals) |
| | thymectacin (NewBiotics) | O6 benzyl guanine (Paligent) |
| Farnesyltransferase | arglabin (NuOncology Labs) | tipifarnib (Johnson & Johnson) |
| inhibitors | lonafarnib (Schering-Plough) | perillyl alcohol (DOR BioPharma) |
| | BAY-43-9006 (Bayer) | |
| Pump inhibitors | CBT-1 (CBA Pharma) | zosuquidar trihydrochloride (Eli Lilly) |
| | tariquidar (Xenova) | biricodar dicitrate (Vertex) |
| | MS-209 (Schering AG) | |
| Histone | tacedinaline (Pfizer) | pivaloyloxymethyl butyrate (Titan) |
| acetyltransferase | SAHA (Aton Pharma) | depsipeptide (Fujisawa) |
| inhibitors | MS-275 (Schering AG) | |
| Metalloproteinase | Neovastat (Aeterna Laboratories) | CMT-3 (CollaGenex) |
| inhibitors | marimastat (British Biotech) | BMS-275291 (Celltech) |
| Ribonucleoside | gallium maltolate (Titan) | tezacitabine (Aventis) |
| reductase inhibitors | triapine (Vion) | didox (Molecules for Health) |
| TNF alpha | virulizin (Lorus Therapeutics) | revimid (Celgene) |
| agonists/antagonists | CDC-394 (Celgene) | |
| Endothelin A | atrasentan (Abbott) | YM-598 (Yamanouchi) |
| receptor antagonist | ZD-4054 (AstraZeneca) | |
| Retinoic acid | fenretinide (Johnson & Johnson) | alitretinoin (Ligand) |
| receptor agonists | LGD-1550 (Ligand) | |
| Immuno- | interferon | dexosome therapy (Anosys) |
| modulators | oncophage (Antigenics) | pentrix (Australian Cancer Technology) |
| | GMK (Progenics) | ISF-154 (Tragen) |
| | adenocarcinoma vaccine (Biomira) | cancer vaccine (Intercell) |
| | CTP-37 (AVI BioPharma) | norelin (Biostar) |
| | IRX-2 (Immuno-Rx) | BLP-25 (Biomira) |
| | PEP-005 (Peplin Biotech) | MGV (Progenics) |
| | synchrovax vaccines (CTL Immuno) | β-alethine (Dovetail) |
| | melanoma vaccine (CTL Immuno) | CLL therapy (Vasogen) |
| | p21 RAS vaccine (GemVax) | |
| Hormonal and | estrogens | dexamethasone |
| antihormonal | conjugated estrogens | prednisone |
| agents | ethinyl estradiol | methylprednisolone |
| | chlortrianisen | prednisolone |
| | idenestrol | aminoglutethimide |
| | hydroxyprogesterone caproate | leuprolide |
| | medroxyprogesterone | octreotide |
| | testosterone | mitotane |
| | testosterone propionate; fluoxymesterone | P-04 (Novogen) |
| | methyltestosterone | 2-methoxyestradiol (EntreMed) |
| | diethylstilbestrol | arzoxifene (Eli Lilly) |
| | megestrol | tamoxifen |
| | bicalutamide | toremofine |
| | flutamide | goserelin |
| | nilutamide | leuporelin |
| Photodynamic | talaporfin (Light Sciences) | Pd-bacteriopheophorbide (Yeda) |
| agents | Theralux (Theratechnologies) | lutetium texaphyrin (Pharmacyclics) |
| | motexafin gadolinium (Pharmacyclics) | hypericin |
| Tyrosine Kinase | imatinib (Novartis) | EKB-569 (Wyeth) |
| Inhibitors | leflunomide (Sugen/Pharmacia) | kahalide F (PharmaMar) |
| | ZD1839 (AstraZeneca) | CEP-701 (Cephalon) |
| | erlotinib (Oncogene Science) | CEP-751 (Cephalon) |
| | canertinib (Pfizer) | MLN518 (Millenium) |
| | squalamine (Genaera) | PKC412 (Novartis) |
| | SU5416 (Pharmacia) | phenoxodiol ( ) |
| | SU6668 (Pharmacia) | C225 (ImClone) |
| | ZD4190 (AstraZeneca) | rhu-Mab (Genentech) |
| | ZD6474 (AstraZeneca) | MDX-H210 (Medarex) |
| | vatalanib (Novartis) | 2C4 (Genentech) |
| | PKI166 (Novartis) | MDX-447 (Medarex) |
| | GW2016 (GlaxoSmithKline) | ABX-EGF (Abgenix) |
| | EKB-509 (Wyeth) | IMC-1C11 (ImClone) |
| | trastuzumab (Genentech) | |
| Miscellaneous agents | SR-27897 (CCK A inhibitor, Sanofi-Synthelabo) | ceflatonin (apoptosis promotor, ChemGenex) |
| | tocladesine (cyclic AMP agonist, Ribapharm) | BCX-1777 (PNP inhibitor, BioCryst) |
| | alvocidib (CDK inhibitor, Aventis) | ranpirnase (ribonuclease stimulant, Alfacell) |
| | CV-247 (COX-2 inhibitor, Ivy Medical) | galarubicin (RNA synthesis inhibitor, Dong-A) |
| | P54 (COX-2 inhibitor, Phytopharm) | tirapazamine (reducing agent, SRI International) |
| | CapCell ™ (CYP450 stimulant, Bavarian Nordic) | N-acetylcysteine (reducing agent, Zambon) |
| | GCS-100 (gal3 antagonist, GlycoGenesys) | R-flurbiprofen (NF-kappaB inhibitor, Encore) |
| | G17DT immunogen (gastrin inhibitor, Aphton) | 3CPA (NF-kappaB inhibitor, Active Biotech) |
| | efaproxiral (oxygenator, Allos Therapeutics) | seocalcitol (vitamin D receptor agonist, Leo) |
| | PI-88 (heparanase inhibitor, Progen) | 131-I-TM-601 (DNA antagonist, TransMolecular) |
| | tesmilifene (histamine antagonist, YM BioSciences) | eflornithine (ODC inhibitor, ILEX Oncology) |
| | histamine (histamine H2 receptor agonist, Maxim) | minodronic acid (osteoclast inhibitor, Yamanouchi) |
| | tiazofurin (IMPDH inhibitor, Ribapharm) | indisulam (p53 stimulant, Eisai) |
| | cilengitide (integrin antagonist, Merck KGaA) | aplidine (PPT inhibitor, PharmaMar) |
| | SR-31747 (IL-1 antagonist, Sanofi-Synthelabo) | gemtuzumab (CD33 antibody, Wyeth Ayerst) |
| | CCI-779 (mTOR kinase inhibitor, Wyeth) | PG2 (hematopoiesis enhancer, Pharmagenesis) |
| | exisulind (PDE V inhibitor, Cell Pathways) | Immunol ™ (triclosan oral rinse, Endo) |
| | CP-461 (PDE V inhibitor, Cell Pathways) | triacetyluridine (uridine prodrug, Wellstat) |
| | AG-2037 (GART inhibitor, Pfizer) | SN-4071 (sarcoma agent, Signature BioScience) |
| | WX-UK1 (plasminogen activator inhibitor, Wilex) | TransMID-107 ™ (immunotoxin, KS Biomedix) |
| | PBI-1402 (PMN stimulant, ProMetic LifeSciences) | PCK-3145 (apoptosis promotor, Procyon) |
| | bortezomib (proteasome inhibitor, Millennium) | doranidazole (apoptosis promotor, Pola) |
| | SRL-172 (T cell stimulant, SR Pharma) | CHS-828 (cytotoxic agent, Leo) |
| | TLK-286 (glutathione S transferase inhibitor, Telik) | trans-retinoic acid (differentiator, NIH) |
| | PT-100 (growth factor agonist, Point Therapeutics) | MX6 (apoptosis promotor, MAXIA) |
| | midostaurin (PKC inhibitor, Novartis) | apomine (apoptosis promotor, ILEX Oncology) |
| | bryostatin-1 (PKC stimulant, GPC Biotech) | urocidin (apoptosis promotor, Bioniche) |
| | CDA-II (apoptosis promotor, Everlife) | Ro-31-7453 (apoptosis promotor, La Roche) |
| | SDX-101 (apoptosis promotor, Salmedix) | brostallicin (apoptosis promotor, Pharmacia) |
| | rituximab (CD20 antibody, Genentech | |

By “Group B antiproliferative agent” is meant any antiproliferative agent selected from the group of compounds in Table 2.

TABLE 2
(Group B)

melphalan
carmustine
Cisplatin
5-fluorouracil
mitomycin C
adriamycin (doxorubicin)
bleomycin
Paclitaxel (Taxol ®)

In one embodiment, the compound of formula (I) is pentamidine, propamidine, butamidine, heptamidine, nonamidine, dibrompropamidine, 1,3-bis(4-amidino-2-methoxyphenoxy)propane, 1,5-bis(4′-(N-hydroxyamidino)phenoxy)pentane, 1,3-bis(4′-(N-hydroxyamidino)phenoxy)propane, 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-O-methylamidoxime, 2,5-bis[4-amidinophenyl]furan-bis-O-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl)thiophene-bis-O-methylamidoxime, 2,4-bis(4-amidinophenyl)thiophene, 2,4-bis(4-amidinophenyl)thiophene-bis-O-methylamidoxime, 2,5-bis[4-(N-isopropylamidino)phenyl]furan, 2,5-bis{4-[3-(dimethylaminopropyl)amidino]phenyl} furan, 2,5-bis{4-[N-(3-aminopropyl)amidino]phenyl}furan, 2,5-bis[4-(2-imidazolinyl)phenyl]-3-methoxyfuran, 2,5-bis[4-(N-isopropylamidino)phenyl]-3-methylfuran, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N,N⁸,N¹¹-trimethylaminopropylcarbamoyl)phenyl]furan, 2,5-bis[3-amidinophenyl]furan, 2,5-bis[3-(N-isopropylamidino)amidinophenyl]furan, 2,5-bis[3[(N-(2-dimethylaminoethyl)amidino]phenylfuran, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis[4-(N-(4-methoxy)phenoxycarbonyl)amidinophenyl] furan, 2,5-bis[4(1-acetoxyethoxycarbonyl)amidinophenyl]furan, or 2,5-bis[4-(N-(3-fluoro)phenoxycarbonyl)amidinophenyl]furan.

In particularly preferred embodiments, the Group A antiproliferative agent is vinblastine, carboplatin, etoposide, or gemcitabine.

In a related aspect, the invention features a method for treating a patient having a cancer or other neoplasm, or inhibiting the development of a neoplasm in a patient who is at risk for developing a neoplasm, by administering to the patient:
(a) a compound of formula (I), depicted above, wherein A is 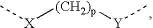
each of X and Y is independently O or NH, and
each of m and n is, independently, an integer between 0 and 2, inclusive, wherein the sum of m and n is greater than 0; and
(b) one or more Group A antiproliferative agents and/or Group B antiproliferative agents.

In another related aspect, the invention features a method for treating a patient having a cancer or other neoplasm, or inhibiting the development of a neoplasm in a patient who is at risk for developing a neoplasm, by administering to the patient (i), a compound having the formula (I), wherein A is 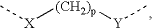
each of X and Y is independently O or NH,
each of m and n is 0, and
each of R¹ and R² is, independently, selected from the group represented by 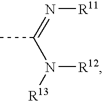
wherein R¹² is C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, carbo(C₁-C₆ alkoxy), carbo(C₆-C₁₈ aryl C₁-C₆ alkoxy), carbo(C₆-C₁₈ aryloxy), or C₆-C₁₈ aryl, and R¹¹ is H, OH, or C₁-C₆ alkyloxy, or R¹¹ and R¹² together represent 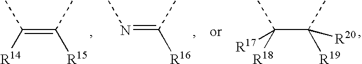
wherein each of R¹⁴, R¹⁵, and R¹⁶ is, independently, H, C₁-C₆ alkyl, halogen, or trifluoromethyl, each of R¹⁷, R¹⁸, and R¹⁹ is, independently, H or C₁-C₆ alkyl, and R²⁰ is C₁-C₆ alkyl, C₁-C₆ alkyloxy, or trifluoromethyl; and
(ii) one or more Group A antiproliferative agents and/or Group B antiproliferative agents.

In yet another related aspect, the invention features a method for treating a patient having a cancer or other neoplasm, or inhibiting the development of a neoplasm in a patient who is at risk for developing a neoplasm, by administering to the patient:
(a) a compound having the formula (I), wherein A is 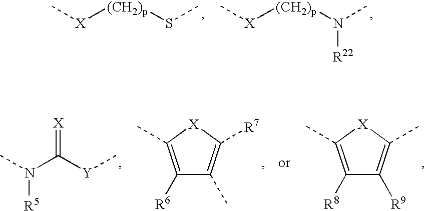
each of X and Y is, independently, O, NR¹⁰, or S,
each of R⁵ and R¹⁰ is, independently, H or C₁-C₆ alkyl,
each of R⁶, R⁷, R⁸, and R⁹ is, independently, H, C₁-C₆ alkyl, halogen, C₁-C₆
alkyloxy, C₆-C₁₈ aryloxy, or C₆-C₁₈ aryl C₁-C₆ alkyloxy,
R²² is C₁-C₆ alkyl,
p is an integer between 2 and 6, inclusive,
each of m and n is, independently, an integer between 0 and 2, inclusive,
each of R¹ and R² is, independently, selected from the group represented by 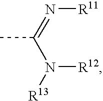
wherein R¹² is H, C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent 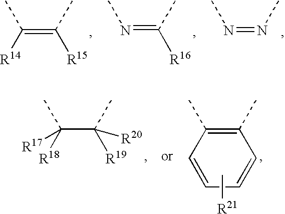
wherein each of R¹⁴, R¹⁵, and R¹⁶ is, independently, H, C₁-C₆ alkyl, halogen, or trifluoromethyl, each of R¹⁷, R¹⁸, R¹⁹, and R²⁰ are, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, or C₆-C₁₈ aryl; and
(b) one or more Group A antiproliferative agents and/or Group B antiproliferative agents.

Preferably, in these related aspects, each antiproliferative agent selected from either Group A or Group B is vinblastine, carboplatin, adriamycin (doxorubicin), etoposide, or gemcitabine

In another aspect, the invention features a method for treating a patient having a cancer or other neoplasm, or inhibiting the development of a neoplasm in a patient who is at risk for developing a neoplasm, by administering to the patient (i), an endo-exonuclease inhibitor, and b), one or more Group A antiproliferative agents.

In another aspect, the invention features a method for treating a patient having a cancer or other neoplasm, or inhibiting the development of a neoplasm in a patient who is at risk for developing a neoplasm, by administering to the patient (a) a phosphatase of regenerating liver (PRL) inhibitor or a PTB1 B inhibitor, and (b) one or more Group A antiproliferative agents.

In either of the two previous aspects, the Group A antiproliferative agent is vinblastine, carboplatin, etoposide, or gemcitabine.

In other aspects, the invention features methods of treating neoplastic cells by contacting the cells with any of the combinations of the aforementioned aspects. In addition, the invention features compositions of compounds used in any of the aspects of the invention, or embodiments thereof.

Components of the combination therapy (e.g., the compound of formula (I), the endo-exonuclease inhibitor, or the PRL inhibitor; and one or more antiproliferative agents) are administered within 14 days of each other in amounts that together are sufficient to inhibit the growth of the neoplasm. Preferably, the components are administered within ten days of each other, more preferably within five days of each other, and most preferably within twenty-four hours of each other or even simultaneously.

Neoplasms treated according to any of the methods of the invention include cancers such as leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, or chronic lymphocytic leukemia), polycythemia vera, lymphoma (Hodgkin's disease or non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, or retinoblastoma). Preferably, the cancer being treated is lung cancer, especially lung cancer attributed to squamous cell carcinoma, adenocarinoma, or large cell carcinoma, colorectal cancer, ovarian cancer, especially ovarian adenocarcinoma, or prostate cancer.

Combination therapy may be provided wherever chemotherapy is performed, such as, for example, at home, the doctor's office, a clinic, a hospital's outpatient department, or a hospital. Treatment generally begins at a hospital so that the doctor can observe the therapy's effects closely and make any adjustments that are needed. The duration of the combination therapy depends on the kind of neoplasm being treated, the age and condition of the patient, the stage and type of the patient's disease, and how the patient's body responds to the treatment. Drug administration may be performed at different intervals (e.g., daily, weekly, or monthly) and the administration of each agent can be determined individually. Combination therapy may be given in on-and-off cycles that include rest periods so that the patient's body has a chance to build healthy new cells and regain its strength.

Depending on the type of cancer and its stage of development, the combination therapy can be used to treat cancer, to slow the spreading of the cancer, to slow the cancer's growth, to kill or arrest cancer cells that may have spread to other parts of the body from the original tumor, to relieve symptoms caused by the cancer, or to prevent cancer in the first place. Combination therapy can also help people live more comfortably by eliminating cancer cells that cause pain or discomfort.

The administration of a combination of the present invention allows for the administration of lower doses of each compound, providing similar efficacy and lower toxicity compared to administration of either compound alone. Alternatively, such combinations result in improved efficacy in treating neoplasms with similar or reduced toxicity.

As used herein, by the terms “cancer” or “neoplasm” or “neoplastic cells” is meant a collection of cells multiplying in an abnormal manner. Cancer growth is uncontrolled and progressive, and occurs under conditions that would not elicit, or would cause cessation of, multiplication of normal cells.

By “inhibits the growth of a neoplasm” is meant measurably slows, stops, or reverses the growth rate of the neoplasm or neoplastic cells in vitro or in vivo. Desirably, a slowing of the growth rate is by at least 20%, 30%, 50%, or even 70%, as determined using a suitable assay for determination of cell growth rates (e.g., a cell growth assay described herein). Typically, a reversal of growth rate is accomplished by initiating or accelerating necrotic or apoptotic mechanisms of cell death in the neoplastic cells, resulting in a shrinkage of the neoplasm.

By “an effective amount” is meant the amount of a compound, in a combination according to the invention, required to inhibit the growth of the cells of a neoplasm in vivo. The effective amount of active compound(s) used to practice the present invention for therapeutic treatment of neoplasms (i.e., cancer) varies depending upon the manner of administration, the age, body weight, and general health of the subject. Ultimately, the attending physician or veterinarian will decide the appropriate amount and dosage regimen. Such amount is referred to as an “effective” amount.

As used herein, the terms “alkyl” and the prefix “alk-” are inclusive of both straight chain and branched chain saturated or unsaturated groups, and of cyclic groups, i.e., cycloalkyl and cycloalkenyl groups. Cyclic groups can be monocyclic or polycyclic and preferably have from 3 to 6 ring carbon atoms, inclusive. Exemplary cyclic groups include cyclopropyl, cyclopentyl, cyclohexyl, and adamantyl groups.

By “carbo(C₁-C₆ alkoxy)” is meant an ester fragment of the structure CO₂R, wherein R is an alkyl group.

By “carbo(C₆-C₁₈ aryl-C₁-C₆ alkoxy)” is meant an ester fragment of the structure CO₂R, wherein R is an alkaryl group.

By “aryl” is meant a C₆-C₁₈ carbocyclic aromatic ring or ring system. Examples of aryl groups include phenyl, naphthyl, biphenyl, fluorenyl, and indenyl groups. The term “heteroaryl” means a C₁-C₉ aromatic ring or ring systems that contains at least one ring heteroatom (e.g., O, S, N). Heteroaryl groups include furyl, thienyl, pyridyl, quinolinyl, tetrazolyl, and imidazolyl groups.

By “halide” or “halogen” is meant bromine, chlorine, iodine, or fluorine.

By “heterocycle” is meant a C₁-C₉ non-aromatic ring or ring system that contains at least one ring heteroatom (e.g., O, S, N). Heterocycles include, for example, pyrrolidinyl, tetrahydrofuranyl, morpholinyl, thiazolidinyl, and imidazolidinyl groups.

Aryl, hetero, and heterocycle groups may be unsubstituted or substituted by one or more substituents selected from the group consisting of C₁₋₆ alkyl, hydroxy, halo, nitro, C₁₋₆ alkoxy, C₁₋₆ alkylthio, trihalomethyl, C₁₋₆ acyl, carbonyl, heteroarylcarbonyl, nitrile, C₁₋₆ alkoxycarbonyl, oxo, alkyl (wherein the alkyl group has from 1 to 6 carbon atoms) and heteroarylalkyl (wherein the alkyl group has from 1 to 6 carbon atoms).

By “endo-exonuclease inhibitor” is meant a compound that inhibits (e.g., by at least 10%, 20%, 30%, or more) the enzymatic activity of an enzyme having endo-exonuclease activity. Such inhibitors include, but are not limited to, pentamidine, pentamidine analogs, and pentamidine metabolites.

By “phosphatase of regenerating liver inhibitor” is meant a compound that inhibits (e.g., by at least 10%, 20%, 30%, or more) the enzymatic activity of a member of the phosphatase of regenerating liver (PRL) family of tyrosine phosphatases. Members of this family include, but are not limited to, PRL-1, PRL-2, and PRL-3. Inhibitors include, but are not limited to, pentamidine, pentamidine analogs, and pentamidine metabolites.

By “protein tyrosine phosphatase 1B inhibitor” is meant a compound that inhibits (e.g., by at least 10%, 20%, 30%, or more) the enzymatic activity of protein phosphatase 1B. Inhibitors include, but are not limited to, pentamidine, pentamidine analogs, and pentamidine metabolites.

Compounds useful in the invention include those described herein in any of their pharmaceutically acceptable forms, including isomers such as diastereomers and enantiomers, salts, solvates, and polymorphs, thereof, as well as racemic mixtures of the compounds described herein.

Other features and advantages of the invention will be apparent from the following detailed description, and from the claims.

### DETAILED DESCRIPTION

The present invention features the combination of the antiprotozoal drug pentamidine with an antiproliferative agent for the treatment or prevention of a neoplasm. Structural and functional analogs of pentamidine are known and, based on known properties that are shared between pentamidine and its analogs and metabolites, any of these analogs or metabolites can be substituted for pentamidine in the antiproliferative combinations of the invention.

Pentamidine

Pentamidine is currently used for the treatment of Pneumocystis carinii, Leishmania donovani, Trypanosoma brucei, T. gambiense, and T. rhodesiense infections. The structure of pentamidine is: 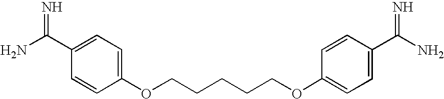
It is available formulated for injection or inhalation. For injection, pentamidine is packaged as a nonpyrogenic, lyophilized product. After reconstitution, it is administered by intramuscular or intravenous injection.

Pentamidine isethionate is a white, crystalline powder soluble in water and glycerin and insoluble in ether, acetone, and chloroform. It is chemically designated 4,4′-diamidino-diphenoxypentane di(β-hydroxyethanesulfonate). The molecular formula is C₂₃H₃₆N₄O₁₀S₂ and the molecular weight is 592.68.

The mode of action of pentamidine is not fully understood. In vitro studies with mammalian tissues and the protozoan Crithidia oncopelti indicate that the drug interferes with nuclear metabolism, producing inhibition of the synthesis of DNA, RNA, phospholipids, and proteins. Several lines of evidence suggest that the action of pentamidine against leishmaniasis, a tropical disease caused by a protozoan residing in host macrophages, might be mediated via host cellular targets and the host immune system. Pentamidine selectively targets intracellular leishmania in macrophages but not the free-living form of the protozoan and has reduced anti-leishmania activity in immunodeficient mice in comparison with its action in immunocompetent hosts.

Recently, pentamidine was shown to be an effective inhibitor of protein tyrosine phosphatase 1B (PTP1 B). Because PTP1 B dephosphorylates and inactivates Jak kinases, which mediate signaling of cytokines with leishmanicidal activity, its inhibition by pentamidine might result in augmentation of cytokine signaling and anti-leishmania effects. Pentamidine has also been shown to be a potent inhibitor of the oncogenic phosphatases of regenerating liver (such as, for example PRL-1, PRL-2, or PRL-3). Thus, in the methods of the invention, pentamidine can be replaced by any protein tyrosine phosphatase inhibitor, including PTP1 B inhibitors or PRL inhibitors. Inhibitors of protein tyrosine phosphatases include levamisole, ketoconazole, bisperoxovanadium compounds (e.g., those described in Scrivens et al., Mol. Cancer Ther. 2:1053-1059, 2003, and U.S. Pat. No. 6,642,221), vandate salts and complexes (e.g., sodium orthovanadate), dephosphatin, dnacin A1, dnacin A2, STI-571, suramin, gallium nitrate, sodium stibogluconate, meglumine antimonate, 2-(2-mercaptoethanol)-3-methyl-1,4-naphthoquinone, 2,5-bis(4-amidinophenyl)furan-bis-O-methylamidoxime, known as DB289 (Immtech), 2,5-bis(4-amidinophenyl)furan (DB75, Immtech), disclosed in U.S. Pat. No. 5,843,980, and compounds described in Pestell et al., Oncogene 19:6607-6612, 2000, Lyon et al., Nat. Rev. Drug Discov. 1:961-976, 2002, Ducruet et al., Bioorg. Med. Chem. 8:1451-1466, 2000, U.S. Patent Application Publication Nos. 2003/0114703, 2003/0144338, 2003/0161893, and PCT Patent Publication Nos. WO99/46237, WO03/06788 and WO03/070158. Still other analogs are those that fall within a formula provided in any of U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, and U.S. Patent Application Publication Nos. US 2001/0044468 and US 2002/0019437, and the pentamidine analogs described in U.S. patent application Ser. No. 10/617,424 (see, e.g., Formula (II)). Other protein tyrosine phosphatase inhibitors can be identified, for example, using the methods described in Lazo et al. (Oncol. Res. 13:347-352, 2003), PCT Publication Nos. WO97/40379, WO03/003001, and WO03/035621, and U.S. Pat. Nos. 5,443,962 and 5,958,719.

Pentamidine has also been shown to inhibit the activity of endo-exonuclease (PCT Publication No. WO 01/35935). Thus, in the methods of the invention, pentamidine can be replaced by any endo-exonuclease inhibitor.

Little is known about the drug's pharmacokinetics. In seven patients treated with daily intramuscular doses of pentamidine at 4 mg/kg for 10 to 12 days, plasma concentrations were between 0.3 and 0.5 μg/mL. The patients continued to excrete decreasing amounts of pentamidine in urine up to six to eight weeks after cessation of the treatment.

Tissue distribution of pentamidine has been studied in mice given a single intraperitoneal injection of pentamidine at 10 mg/kg. The concentration in the kidneys was the highest, followed by that in the liver. In mice, pentamidine was excreted unchanged, primarily via the kidneys with some elimination in the feces. The ratio of amounts excreted in the urine and feces (4:1) was constant over the period of study.

Pentamidine Analogs

Aromatic diamidino compounds can replace pentamidine in the antiproliferative combination of the invention. Aromatic diamidino compounds such as propamidine, butamidine, heptamidine, and nonamidine share properties with pentamidine in that they exhibit antipathogenic or DNA binding properties. Other analogs (e.g., stilbamidine and indole analogs of stilbamidine, hydroxystilbamidine, diminazene, benzamidine, 4,4′-(pentamethylenedioxy)phenamidine, dibrompropamidine, 1,3-bis(4-amidino-2-methoxyphenoxy)propane (DAMP), netropsin, distamycin, phenamidine, amicarbalide, bleomycin, actinomycin, and daunorubicin) also exhibit properties similar to those of pentamidine. It is likely that these compounds will have anti-cancer activity when administered in combination with an antiproliferative agent.

Pentamidine analogs are described, for example, by formula (I): 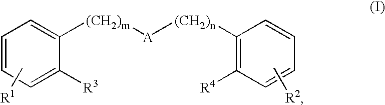
or a pharmaceutically acceptable salt thereof,
wherein A is 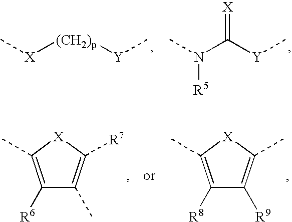
wherein
each of X and Y is, independently, O, NR¹⁰, or S,
each of R⁵ and R¹⁰ is, independently, H or C₁-C₆ alkyl,
each of R⁶, R⁷, R⁸, and R⁹ is, independently, H, C₁-C₆ alkyl, halogen, C₁-C₆
alkyloxy, C₆-C₁₈ aryloxy, or C₆-C₁₈ aryl-C₁-C₆ alkyloxy,
p is an integer between 2 and 6, inclusive,
each of m and n is, independently, an integer between 0 and 2, inclusive,
each of R¹ and R² is 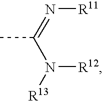
wherein R¹² is H, C₁-C₆ alkyl, C₁-C₈ cycloalkyl, C₁-C₆ alkyloxy-C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent 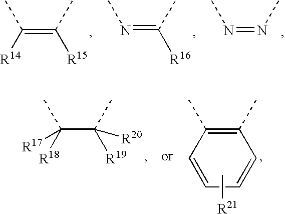
wherein each of R¹⁴, R¹⁵, and R¹⁶ is, independently, H, C₁-C₆ alkyl, halogen, or trifluoromethyl, each of R¹⁷, R¹⁸, R¹⁹, and R²⁰ is, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl,
each of R³ and R⁴ is, independently, H, Cl, Br, OH, OCH₃, OCF₃, NO₂, and NH₂, or R³ and R⁴ together form a single bond.

Other analogs include stilbamidine (G-1) and hydroxystilbamidine (G-2), and their indole analogs (e.g., G-3).

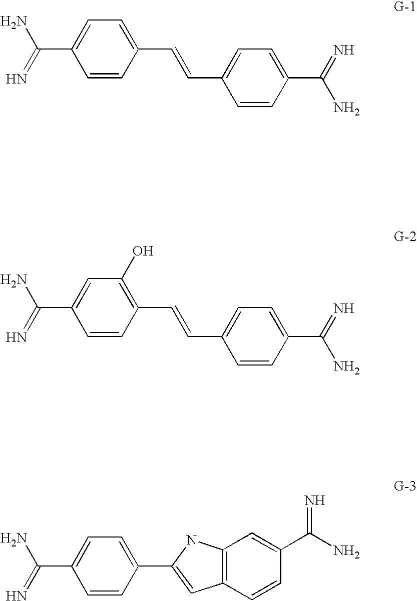

Each amidine moiety in G-1, G-2, or G-3 may be replaced with one of the moieties depicted in formula (I) above as

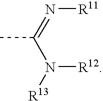

As is the case for pentamidine, salts of stilbamidine and its related compounds are also useful in the method of the invention. Preferred salts include, for example, dihydrochloride and methanesulfonate salts.

Still other analogs are those that fall within a formula provided in any of U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, or U.S. Patent Application Publication Nos. US 2001/0044468 A1 and US 2002/0019437 A1, each of which is in its entirety incorporated by reference.

Exemplary analogs are 1,3-bis(4-amidino-2-methoxyphenoxy)propane, phenamidine, amicarbalide, 1,5-bis(4′-(N-hydroxyamidino)phenoxy)pentane, 1,3-bis(4′-(N-hydroxyamidino)phenoxy)propane, 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-O-methylamidoxime, 2,5-bis[4-amidinophenyl]furan-bis-O-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl)thiophene-bis-O-methylamidoxime, 2,4-bis(4-amidinophenyl)thiophene, 2,4-bis(4-amidinophenyl)thiophene-bis-O-methylamidoxime, 2,8-diamidinodibenzothiophene, 2,8-bis(N-isopropylamidino)carbazole, 2,8-bis(N-hydroxyamidino)carbazole, 2,8-bis(2-imidazolinyl)dibenzothiophene, 2,8-bis(2-imidazolinyl)-5,5-dioxodibenzothiophene, 3,7-diamidinodibenzothiophene, 3,7-bis(N-isopropylamidino)dibenzothiophene, 3,7-bis(N-hydroxyamidino)dibenzothiophene, 3,7-diaminodibenzothiophene, 3,7-dibromodibenzothiophene, 3,7-dicyanodibenzothiophene, 2,8-diamidinodibenzofuran, 2,8-di(2-imidazolinyl)dibenzofuran, 2,8-di(N-isopropylamidino)dibenzofuran, 2,8-di(N-hydroxylamidino)dibenzofuran, 3,7-di(2-imidazolinyl)dibenzofuran, 3,7-di(isopropylamidino)dibenzofuran, 3,7-di(N-hydroxylamidino)dibenzofuran, 2,8-dicyanodibenzofuran, 4,4′-dibromo-2,2′-dinitrobiphenyl, 2-methoxy-2′-nitro-4,4′-dibromobiphenyl, 2-methoxy-2′-amino-4,4′-dibromobiphenyl, 3,7-dibromodibenzofuran, 3,7-dicyanodibenzofuran, 2,5-bis(5-amidino-2-benzimidazolyl)pyrrole, 2,5-bis[5-(2-imidazolinyl)-2-benzimidazolyl]pyrrole, 2,6-bis[5-(2-imidazolinyl)-2-benzimidazolyl]pyridine, 1-methyl-2,5-bis(5-amidino-2-benzimidazolyl)pyrrole, 1-methyl-2,5-bis[5-(2-imidazolyl)-2-benzimidazolyl]pyrrole, 1-methyl-2,5-bis[5-(1,4,5,6-tetrahydro-2-pyrimidinyl)-2-benzimidazolyl]pyrrole, 2,6-bis(5-amidino-2-benzimidazoyl)pyridine, 2,6-bis[5-(1,4,5,6-tetrahydro-2-pyrimidinyl)-2-benzimidazolyl]pyridine, 2,5-bis(5-amidino-2-benzimidazolyl)furan, 2,5-bis-[5-(2-imidazolinyl)-2-benzimidazolyl]furan, 2,5-bis-(5-N-isopropylamidino-2-benzimidazolyl)furan, 2,5-bis-(4-guanylphenyl)furan, 2,5-bis(4-guanylphenyl)-3,4-dimethylfuran, 2,5-bis{p-[2-(3,4,5,6-tetrahydropyrimidyl)phenyl]}furan, 2,5-bis[4-(2-imidazolinyl)phenyl]furan, 2,5 [bis-{4-(2-tetrahydropyrimidinyl)}phenyl]-3-(p-tolyloxy)furan, 2,5 [bis {4-(2-imidazolinyl)}phenyl]-3-(p-tolyloxy)furan, 2,5-bis{4-[5-(N-2-aminoethylamido)benzimidazol-2-yl]phenyl}furan, 2,5-bis[4-(3a,4,5,6,7,7a-hexahydro-1H-benzimidazol-2-yl)phenyl]furan, 2,5-bis [4-(4,5,6,7-tetrahydro-1H-1,3-diazepin-2-yl)phenyl]furan, 2,5-bis(4-N,N-dimethylcarboxhydrazidephenyl)furan, 2,5-bis {4-[2-(N-2-hydroxyethyl)imidazolinyl]phenyl}furan, 2,5-bis[4-(N-isopropylamidino)phenyl]furan, 2,5-bis{4-[3-(dimethylaminopropyl)amidino]phenyl}furan, 2,5-bis {4-[N-(3-aminopropyl)amidino]phenyl}furan, 2,5-bis[2-(imidzaolinyl)phenyl]-3,4-bis(methoxymethyl)furan, 2,5-bis[4-N-(dimethylaminoethyl)guanyl]phenylfuran, 2,5-bis {4-[(N-2-hydroxyethyl)guanyl]phenyl}furan, 2,5-bis[4-N-(cyclopropylguanyl)phenyl]furan, 2,5-bis[4-(N,N-diethylaminopropyl)guanyl]phenylfuran, 2,5-bis{4-[2-(N-ethylimidazolinyl)]phenyl}furan, 2,5-bis {4-[N-(3-pentylguanyl)]}phenylfuran, 2,5-bis[4-(2-imidazolinyl)phenyl]-3-methoxyfuran, 2,5-bis[4-(N-isopropylamidino)phenyl]-3-methylfuran, bis[5-amidino-2-benzimidazolyl]methane, bis[5-(2-imidazolyl)-2-benzimidazolyl]methane, 1,2-bis[5-amidino-2-benzimidazolyl]ethane, 1,2-bis[5-(2-imidazolyl)-2-benzimidazolyl]ethane, 1,3-bis[5-amidino-2-benzimidazolyl]propane, 1,3-bis[5-(2-imidazolyl)-2-benzimidazolyl]propane, 1,4-bis[5-amidino-2-benzimidazolyl]propane, 1,4-bis[5-(2-imidazolyl)-2-benzimidazolyl]butane, 1,8-bis[5-amidino-2-benzimidazolyl]octane, trans-1,2-bis[5-amidino-2-benzimidazolyl]ethene, 1,4-bis[5-(2-imidazolyl)-2-benzimidazolyl]-1-butene, 1,4-bis[5-(2-imidazolyl)-2-benzimidazolyl]-2-butene, 1,4-bis[5-(2-imidazolyl)-2-benzimidazolyl]-1-methylbutane, 1,4-bis[5-(2-imidazolyl)-2-benzimidazolyl]-2-ethylbutane, 1,4-bis[5-(2-imidazolyl)-2-benzimidazolyl]-1-methyl-1-butene, 1,4-bis[5-(2-imidazolyl)-2-benzimidazolyl]-2,3-diethyl-2-butene, 1,4-bis[5-(2-imidazolyl)-2-benzimidazolyl]-1,3-butadiene, 1,4-bis[5-(2-imidazolyl)-2-benzimidazolyl]-2-methyl-1,3-butadiene, bis[5-(2-pyrimidyl)-2-benzimidazolyl]methane, 1,2-bis[5-(2-pyrimidyl)-2-benzimidazolyl]ethane, 1,3-bis[5-amidino-2-benzimidazolyl]propane, 1,3-bis[5-(2-pyrimidyl)-2-benzimidazolyl]propane, 1,4-bis[5-(2-pyrimidyl)-2-benzimidazolyl]butane, 1,4-bis[5-(2-pyrimidyl)-2-benzimidazolyl]-1-butene, 1,4-bis[5-(2-pyrimidyl)-2-benzimidazolyl]-2-butene, 1,4-bis[5-(2-pyrimidyl)-2-benzimidazolyl]-1-methylbutane, 1,4-bis[5-(2-pyrimidyl)-2-benzimidazolyl]-2-ethylbutane, 1,4-bis[5-(2-pyrimidyl)-2-benzimidazolyl]-1-methyl-1-butene, 1,4-bis[5-(2-pyrimidyl)-2-benzimidazolyl]-2,3-diethyl-2-butene, 1,4-bis[5-(2-pyrimidyl)-2-benzimidazolyl]-1,3-butadiene, and 1,4-bis[5-(2-pyrimidyl)-2-benzimidazolyl]-2-methyl-1,3-butadiene, 2,4-bis(4-guanylphenyl)pyrimidine, 2,4-bis(4-imidazolin-2-yl)pyrimidine, 2,4-bis[(tetrahydropyrimidinyl-2-yl)phenyl]pyrimidine, 2-(4-[N-1-propylguanyl]phenyl)-4-(2-methoxy-4-[N-1-propylguanyl]phenyl)pyrimidine, 4-(N-cyclopentylamidino)-1,2-phenylene diamine, 2,5-bis-[2-(5-amidino)benzimidazoyl]furan, 2,5-bis[2-{5-(2-imidazolino)}benzimidazoyl]furan, 2,5-bis[2-(5-N-isopropylamidino)benzimidazoyl]furan, 2,5-bis[2-(5-N-cyclopentylamidino)benzimidazoyl]furan, 2,5-bis[2-(5-amidino)benzimidazoyl]pyrrole, 2,5-bis[2-{5-(2-imidazolino)}benzimidazoyl]pyrrole, 2,5-bis[2-(5-N-isopropylamidino)benzimidazoyl]pyrrole, 2,5-bis[2-(5-N-cyclopentylamidino)benzimidazoyl]pyrrole, 1-methyl-2,5-bis[2-(5-amidino)benzimidazoyl]pyrrole, 2,5-bis[2-{5-(2-imidazolino)}benzimidazoyl]-1-methylpyrrole, 2,5-bis[2-(5-N-cyclopentylamidino)benzimidazoyl]-1-methylpyrrole, 2,5-bis[2-(5-N-isopropylamidino)benzimidazoyl]thiophene, 2,6-bis[2-{5-(2-imidazolino)}benzimidazoyl]pyridine, 2,6-bis[2-(5-amidino)benzimidazoyl]pyridine, 4,4′-bis[2-(5-N-isopropylamidino)benzimidazoyl]-1,2-diphenylethane, 4,4′-bis[2-(5-N-cyclopentylamidino)benzimidazoyl]-2,5-diphenylfuran, 2,5-bis[2-(5-amidino)benzimidazoyl]benzo[b]furan, 2,5-bis[2-(5-N-cyclopentylamidino)benzimidazoyl]benzo[b]furan, 2,7-bis[2-(5-N-isopropylamidino)benzimidazoyl]fluorene, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N,N⁸,N¹¹-trimethylaminopropylcarbamoyl)phenyl]furan, 2,5-bis [3-amidinophenyl]furan, 2,5-bis[3-(N-isopropylamidino)amidinophenyl]furan, 2,5-bis[3 [(N-(2-dimethylaminoethyl)amidino]phenylfuran, 2,5-bis[4-(N-2,2,2-trichloroethoxycarbonyl)amidinophenyl]furan, 2,5-bis[4-(N-thioethylcarbonyl) amidinophenyl]furan, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis [4-(N-(4-methoxy)phenoxycarbonyl)amidinophenyl]furan, 2,5-bis[4(1-acetoxyethoxycarbonyl)amidinophenyl]furan, and 2,5-bis [4-(N-(3-fluoro)phenoxycarbonyl)amidinophenyl]furan. Methods for making any of the foregoing compounds are described in U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, an U.S. Patent Application Publication Nos. US 2001/0044468 A1 and US 2002/0019437 A1.

Pentamidine Metabolites

Pentamidine metabolites are also useful in the antiproliferative combination of the invention. Pentamidine is rapidly metabolized in the body to at least seven primary metabolites. Some of these metabolites share one or more activities with pentamidine. It is likely that some pentamidine metabolites will have anti-cancer activity when administered in combination with an antiproliferative agent. Seven pentamidine metabolites (H-1 through H-7) are shown below. 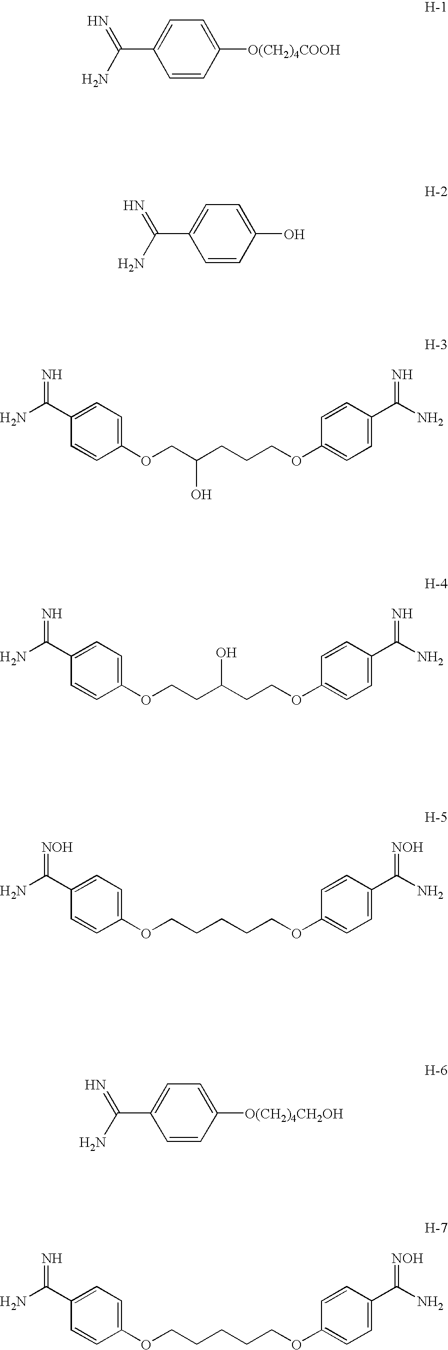
Therapy

The combinations of the invention are useful for the treatment of neoplasms. Therapy may be performed alone or in conjunction with another therapy (e.g., surgery, radiation therapy, immunotherapy, or gene therapy). Additionally, a person having a greater risk of developing a neoplasm (e.g., one who is genetically predisposed or one who previously had a neoplasm) may receive prophylactic treatment to inhibit or delay neoplastic formation. The duration of the combination therapy depends on the type of disease or disorder being treated, the age and condition of the patient, the stage and type of the patient's disease, and how the patient responds to the treatment. Therapy may be given in on-and-off cycles that include rest periods so that the patient's body has a chance to recovery from any as yet unforeseen side-effects.

Examples of cancers and other neoplasms include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenriglioma, schwannoma, meningioma, melanoma, neuroblastoma, or retinoblastoma).

Formulation of Pharmaceutical Compositions

The administration of each compound of the combination may be by any suitable means that results in a concentration of the compound that, combined with the other component, is anti-neoplastic upon reaching the target region. The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously or intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Dosages

The dosage of each compound or agent of the claimed combinations depends on several factors, including: the administration method, the neoplasm to be treated, the severity of the neoplasm, whether the neoplasm is to be treated or prevented, and the age, weight, and health of the patient to be treated.

The compound or agent in question may be administered orally in the form of tablets, capsules, elixirs or syrups, or rectally in the form of suppositories. Parenteral administration of a compound is suitably performed, for example, in the form of saline solutions or with the compound incorporated into liposomes. In cases where the compound in itself is not sufficiently soluble to be dissolved, a solubilizer such as ethanol can be applied. An antiproliferative agent of the invention is usually given by the same route of administration that is known to be effective for delivering it as a monotherapy. When used in combination therapy with pentamidine or a pentamidine analog according to the methods of this invention, the antiproliferative agent is dosed in amounts and frequencies equivalent to or less than those that result in its effective monotherapeutic use.

Pentamidine Dosage

For pentamidine or a pentamidine analog, oral dosage is normally about 0.1 mg to 300 mg per dose administered (preferably about 1 mg to 100 mg) one to four times daily for one day to one year and may be administered for the life of the patient. Administration may also be given in cycles, such that there are periods during which time pentamidine is not administered. This period could be, for example, about a day, a week, a month, or a year or more.

The rectal dosage of pentamidine or a pentamidine analog is as described for orally administered pentamidine.

For intravenous or intramuscular administration of pentamidine or a pentamidine analog, a daily dose of about 0.05 mg/kg to about 20 mg/kg is recommended, a dose of about 0.05 mg/kg to about 10 mg/kg is preferred, and a dose of about 0.1 mg/kg to about 4 mg/kg is most preferred. Intravenous or intramuscular administration is usually daily for up to about 6 to 12 months or more. It may be desirable to administer a compound over a one to three hour period; this period may be extended to last 24 hours or more. As is described for oral administration, there may be periods of about one day to one year or longer during which at least one of the drugs is not administered.

For inhalation, pentamidine or a pentamidine analog is administered at a dose of about 1 mg to 1000 mg, and preferably at a dose of 2 mg to 600 mg, is administered daily.

For topical administration of pentamidine or a pentamidine analog, a dose of about 1 mg to about 5 g administered one to ten times daily for one week to 12 months is usually preferable.

### EXAMPLES

Tumor Cell Culture

Human non-small cells lung carcinoma cells A549 (ATCC# CCL-185), were grown at 37±0.5° C. and 5% CO₂ in DMEM supplemented with 10% FBS, 2 mM glutamine, 1% penicillin, and 1% streptomycin.

Test Compounds

Pentamidine, 5-fluorouracil, carboplatin, doxorubicin, etoposide, gemcitabine, and vinblastine were obtained from Sigma Chemical Co. (St. Louis, Mo.). Stock solutions (1000×) of each compound were prepared in DMSO and stored at −20° C. Master stock plates of 2-fold or 4-fold serial dilutions of individual compounds were prepared in 384-well plates. Combination matrices of test compounds were generated from these master stock plates by dilution into growth media described above. The final concentration of test compounds in the combination matrices was 10× greater than used in the assay. The combination matrices were used immediately and discarded.

Anti-Proliferation Assay

The anti-proliferation assays were performed in 384-well plates. 6.6 μL of 10× stock solutions from the combination matrices were added to 40 μL of culture media in assay wells. The tumor cells were liberated from the culture flask using a solution of 0.25% trypsin. Cells were diluted in culture media such that 3000 or 6000 cells were delivered in 20 μL of media into each assay well. Assay plates were incubated for 72-80 hours at 37° C. ±0.5° C. with 5% CO₂. Twenty microliters of 20% Alamar Blue warmed to 37° C. ±0.5° C. was added to each assay well following the incubation period. Alamar Blue metabolism was quantified by the amount of fluorescence intensity 3.5-5.0 hours after addition. Quantification, using an LJL Analyst AD reader (LJL Biosystems), was taken in the middle of the well with high attenuation, a 100 msec read time, an excitation filter at 530 nm, and an emission filter at 575 nm. For some experiments, quantification was performed using a Wallac Victor² reader. Measurements were taken at the top of the well with stabilized energy lamp control; a 100 msec read time, an excitation filter at 530 nm, and an emission filter at 590 nm. No significant differences between plate readers were measured.

The percent inhibition (% I) for each well was calculated using the following formula:
% I=[(avg. untreated wells−treated well)/(avg. untreated wells)]×100
The average untreated well value (avg. untreated wells) is the arithmetic mean of 40 wells from the same assay plate treated with vehicle alone. Negative inhibition values result from local variations in treated wells as compared to untreated wells.

### Example 1

### Antiproliferative Activity of Pentamidine and Vinblastine Against Non-Small Cell Lung Carcinoma A549 Cells

Inhibition of proliferation was measured by anti-proliferation assay as described below after incubation with the test compound(s) for 72 hours. The effects of varying concentrations of pentamidine, vinblastine, or a combination of pentamidine and vinblastine were compared to control wells (seeded with A549 cells, but not incubated with either pentamidine or vinblastine).

The results of this experiment are shown in Table 3. The effects of the agents alone and in combination are shown as percent inhibition of cell proliferation.

TABLE 3
Percent inhibition of Alamar Blue Metabolism in A549 cells

| | Pentamidine (μM) | | | | | | | | | |
|---|---|---|---|---|---|---|---|---|---|---|
| Vinblastine (μM) | 3.374 | 1.687 | 0.844 | 0.422 | 0.211 | 0.105 | 0.053 | 0.026 | 0.013 | 0.000 |
| 0.0250 | 91.0 | 88.5 | 88.8 | 88.1 | 87.9 | 86.3 | 85.7 | 85.8 | 85.5 | 85.5 |
| 0.0125 | 90.9 | 88.5 | 86.7 | 84.2 | 80.8 | 81.7 | 79.7 | 79.7 | 74.5 | 77.2 |
| 0.0063 | 90.0 | 83.0 | 80.7 | 77.7 | 74.2 | 68.6 | 69.3 | 71.1 | 68.4 | 67.2 |
| 0.0031 | 89.5 | 84.3 | 79.7 | 77.6 | 65.8 | 59.7 | 55.7 | 56.7 | 62.3 | 61.1 |
| 0.0016 | 89.2 | 79.9 | 71.4 | 64.8 | 51.7 | 45.5 | 38.8 | 12.0 | 45.4 | 48.4 |
| 0.0008 | 86.6 | 77.1 | 65.7 | 53.3 | 25.6 | 15.1 | 19.1 | −7.4 | 31.5 | 41.5 |
| 0.0004 | 85.7 | 74.5 | 63.6 | 49.3 | 18.2 | 3.0 | 8.3 | −3.7 | 5.4 | 7.4 |
| 0.0002 | 86.3 | 74.9 | 67.5 | 59.4 | 25.9 | 7.6 | −7.9 | −11.9 | −8.7 | −8.3 |
| 0.0001 | 86.1 | 77.3 | 67.9 | 44.9 | 27.2 | −11.5 | −15.7 | −21.5 | −14.6 | −22.3 |
| 0.0000 | 85.4 | 78.2 | 71.9 | 52.1 | 19.3 | 4.7 | −12.4 | −25.3 | −28.2 | −25.6 |

### Example 2

### Antiproliferative Activity of Pentamidine and Carboplatin Against A549 Cells

Table 4 shows the results from an anti-proliferation assay using A549cells treated with pentamidine, carboplatin, or a combination of pentamidine and carboplatin.

TABLE 4
Percent inhibition of Alamar Blue Metabolism in A549 cells

| | Pentamidine (μM) | | | | | | | | | |
|---|---|---|---|---|---|---|---|---|---|---|
| Carboplatin (μM) | 3.370 | 1.685 | 0.843 | 0.421 | 0.211 | 0.105 | 0.053 | 0.026 | 0.013 | 0.000 |
| 38.00 | 82.0 | 80.3 | 76.5 | 65.4 | 56.5 | 45.0 | 43.3 | 50.4 | 49.8 | 48.7 |
| 19.00 | 81.4 | 77.1 | 68.7 | 59.2 | 43.7 | 45.3 | 34.5 | 26.4 | 35.5 | 30.5 |
| 9.50 | 78.0 | 79.9 | 70.1 | 76.3 | 54.4 | 15.8 | 34.2 | 25.7 | 35.2 | 35.8 |
| 4.75 | 85.3 | 77.4 | 73.9 | 51.1 | 25.2 | 56.1 | 51.7 | 4.3 | 32.3 | 16.4 |
| 2.38 | 84.2 | 30.1 | 72.4 | 12.3 | 21.1 | 16.1 | 2.5 | 21.1 | −4.8 | 13.9 |
| 1.19 | 84.4 | 81.4 | 49.8 | 69.4 | 52.0 | 17.8 | 11.5 | −11.5 | 1.1 | −19.0 |
| 0.59 | 82.0 | 73.8 | 72.4 | 61.7 | 35.6 | −3.8 | −16.1 | −0.7 | 11.5 | 9.8 |
| 0.30 | 80.0 | 77.4 | 79.7 | 53.7 | 13.5 | −34.7 | 20.9 | 19.3 | −12.8 | 14.5 |
| 0.15 | 76.6 | 81.0 | 72.2 | 51.0 | 26.6 | 12.1 | 5.9 | 5.9 | −23.2 | 5.5 |
| 0.00 | 79.4 | 79.1 | 78.2 | 50.0 | 30.6 | −3.7 | −8.8 | −6.8 | 8.4 | −5.9 |

### Example 3

### Antiproliferative Activity of Pentamidine and Doxorubicin Against Human A549 Cells

The results from a 2-fold dilution series of pentamidine and doxorubicin combination on A549 cell growth are shown in Table 5.

TABLE 5
Percent inhibition of Alamar Blue Metabolism in A549 cells

| | Pentamidine (μM) | | | | | | | | | |
|---|---|---|---|---|---|---|---|---|---|---|
| Doxorubicin (μM) | 3.374 | 1.687 | 0.844 | 0.422 | 0.211 | 0.105 | 0.053 | 0.026 | 0.013 | 0.000 |
| 0.2000 | 87.6 | 75.7 | 74.3 | 79.6 | 81.8 | 57.5 | 59.0 | 60.1 | 65.5 | 62.9 |
| 0.1000 | 86.3 | 81.4 | 72.2 | 67.9 | 71.6 | 60.5 | 60.1 | 58.8 | 62.7 | 63.0 |
| 0.0500 | 91.7 | 82.9 | 76.8 | 71.8 | 69.8 | 60.2 | 59.7 | 63.1 | 74.0 | 67.5 |
| 0.0250 | 88.1 | 83.9 | 79.3 | 71.5 | 70.4 | 53.1 | 57.7 | 63.1 | 59.7 | 63.8 |
| 0.0125 | 86.7 | 82.8 | 72.4 | 66.2 | 64.1 | 41.2 | 39.0 | 46.0 | 64.3 | 55.6 |
| 0.0063 | 86.6 | 78.2 | 78.5 | 69.9 | 67.5 | 40.9 | 44.1 | 40.3 | 33.2 | 40.3 |
| 0.0031 | 85.6 | 78.3 | 70.9 | 62.4 | 49.4 | 31.8 | 31.1 | 32.7 | 31.6 | 34.4 |
| 0.0016 | 88.1 | 78.7 | 71.9 | 59.8 | 57.8 | 37.2 | 36.1 | 30.1 | 30.2 | 27.5 |
| 0.0008 | 85.6 | 82.7 | 79.8 | 61.2 | 45.5 | 34.2 | 30.9 | 27.8 | 29.4 | 32.0 |
| 0.0000 | 87.5 | 83.1 | 80.0 | 68.2 | 49.6 | 33.7 | 30.4 | 28.4 | 30.2 | 34.0 |

### Example 4

### Antiproliferative Activity of Pentamidine and Etoposide Against A549 Cells

The results from a 2-fold dilution series of pentamidine and etoposide combination on A549 cell growth are shown in Table 6.

TABLE 6
Percent inhibition of Alamar Blue Metabolism in A549 cells

| | Etoposide (μM) | | | | | | | | | |
|---|---|---|---|---|---|---|---|---|---|---|
| Pentamidine (μM) | 10.00 | 5.00 | 2.50 | 1.25 | 0.63 | 0.31 | 0.16 | 0.08 | 0.04 | 0.00 |
| 3.370 | 89.1 | 87.6 | 85.4 | 83.9 | 82.5 | 82.2 | 81.8 | 79.7 | 79.4 | 80.6 |
| 1.685 | 87.2 | 83.3 | 81.2 | 80.9 | 77.1 | 74.6 | 72.7 | 70.7 | 71.2 | 71.9 |
| 0.843 | 85.0 | 80.9 | 79.5 | 73.2 | 71.9 | 67.2 | 63.9 | 67.3 | 58.3 | 59.3 |
| 0.421 | 84.1 | 75.4 | 69.7 | 65.9 | 56.6 | 46.8 | 45.0 | 36.0 | 27.3 | 32.6 |
| 0.211 | 85.8 | 78.3 | 71.6 | 72.3 | 59.0 | 44.9 | 36.8 | 17.4 | 4.0 | 5.0 |
| 0.105 | 80.3 | 73.3 | 73.2 | 64.1 | 52.7 | 36.9 | 22.9 | 7.1 | −6.6 | −3.7 |
| 0.053 | 88.5 | 87.0 | 84.9 | 82.7 | 78.0 | 74.9 | 56.6 | 35.6 | 27.9 | 1.1 |
| 0.026 | 88.6 | 83.2 | 80.1 | 80.8 | 82.3 | 77.0 | 60.2 | 54.6 | 17.8 | 13.1 |
| 0.013 | 81.6 | 83.4 | 75.6 | 70.2 | 73.9 | 60.2 | 50.8 | 25.4 | 8.3 | −6.7 |
| 0.000 | 79.4 | 77.5 | 76.1 | 62.8 | 52.4 | 42.7 | 38.7 | 46.5 | 3.9 | 4.8 |

### Example 5

### Antiproliferative Activity of Pentamidine and Gemcitabine Against A549 Cells

The results from a 2-fold dilution series of pentamidine and gemcitabine combination on A549 cell growth are shown in Table 7.

TABLE 7
Percent inhibition of Alamar Blue Metabolism in A659 cells

| | Pentamidine (μM) | | | | | | | | | |
|---|---|---|---|---|---|---|---|---|---|---|
| Gemcitabine (μM) | 3.370 | 1.685 | 0.843 | 0.421 | 0.211 | 0.105 | 0.053 | 0.026 | 0.013 | 0.000 |
| 0.04200 | 91.8 | 91.1 | 89.7 | 87.7 | 87.4 | 89.6 | 89.1 | 88.7 | 88.3 | 89.8 |
| 0.02100 | 90.8 | 89.1 | 87.3 | 85.5 | 87.8 | 88.2 | 87.5 | 87.6 | 89.3 | 88.7 |
| 0.01050 | 87.8 | 86.2 | 81.3 | 80.3 | 85.1 | 89.0 | 86.0 | 86.3 | 84.5 | 87.4 |
| 0.00525 | 82.7 | 78.4 | 69.7 | 61.5 | 77.5 | 61.6 | 49.4 | 59.2 | 62.0 | 61.8 |
| 0.00263 | 74.0 | 70.4 | 43.5 | 47.7 | 34.5 | 18.1 | 57.5 | 26.8 | 38.1 | 13.8 |
| 0.00131 | 71.6 | 62.6 | 34.6 | 28.2 | 11.9 | 8.4 | 30.2 | 24.6 | 22.0 | 12.3 |
| 0.00066 | 78.4 | 68.8 | 46.7 | 8.1 | 5.8 | 11.0 | 21.8 | 34.5 | 33.0 | 11.3 |
| 0.00033 | 75.9 | 71.1 | 50.6 | 8.3 | 5.2 | 10.3 | 16.2 | 0.9 | 16.9 | 19.1 |
| 0.00016 | 65.2 | 56.1 | 25.6 | 3.1 | −3.5 | −7.2 | 3.8 | −10.7 | 2.0 | 7.9 |
| 0.00000 | 73.7 | 62.9 | 38.6 | 10.5 | −4.2 | 9.2 | −1.9 | −3.0 | −4.4 | 2.2 |

### Example 6

### Antiproliferative Activity of Pentamidine and 5-Fluorouracil Against A549 Cells

The results from a non-linear dilution series of a pentamidine and 5-fluorouracil combination on A549 cell growth are shown in Table 8.

TABLE 8
Percent inhibition of Alamar Blue Metabolism in A549 cells

| | Pentamidine (μM) | | | | | | | | | |
|---|---|---|---|---|---|---|---|---|---|---|
| 5-Fluorouracil (μM) | 1.700 | 1.300 | 1.000 | 0.700 | 0.500 | 0.350 | 0.200 | 0.100 | 0.050 | 0.000 |
| 10.00 | 83.0 | 84.0 | 81.1 | 82.9 | 82.3 | 81.4 | 81.2 | 82.9 | 80.6 | 82.4 |
| 5.00 | 80.8 | 81.8 | 76.6 | 79.0 | 78.4 | 79.3 | 78.7 | 78.4 | 78.8 | 79.8 |
| 2.00 | 82.5 | 80.3 | 82.8 | 81.2 | 83.5 | 83.5 | 71.8 | 83.9 | 71.5 | 87.2 |
| 1.50 | 90.9 | 76.9 | 90.0 | 65.8 | 81.6 | 74.5 | 88.7 | 74.5 | 74.8 | 73.6 |
| 1.00 | 89.0 | 88.6 | 87.6 | 80.2 | 72.4 | 81.9 | 74.0 | 82.5 | 73.3 | 75.4 |
| 0.75 | 90.8 | 87.8 | 87.2 | 73.6 | 70.7 | 55.8 | 68.6 | 60.6 | 75.0 | 64.3 |
| 0.50 | 84.0 | 86.9 | 60.4 | 79.7 | 52.7 | 54.5 | 12.4 | 52.9 | 34.4 | 40.6 |
| 0.10 | 79.3 | 74.8 | 67.4 | 41.3 | 29.5 | −7.0 | 18.8 | −8.3 | −1.8 | −0.5 |
| 0.01 | 71.8 | 70.6 | 44.8 | 32.7 | 9.1 | 2.5 | −6.9 | −2.3 | −0.4 | 2.9 |
| 0.00 | 70.1 | 64.7 | 48.9 | 21.7 | 15.8 | −1.5 | −0.7 | −3.1 | 2.4 | 10.2 |

Other Embodiments

The anti-proliferative effect demonstrated with the tumor cell lines used herein can be similarly demonstrated using other cancer cell lines, such as NSC lung carcinoma, MCF7 mammary adenocarcinoma, PA-1 ovarian teratocarcinoma, HT29 colorectal adenocarcinoma, H1299 large cell carcinoma, U-2 OS osteogenic sarcoma, U-373 MG glioblastoma, Hep-3B hepatocellular carcinoma, BT-549 mammary carcinoma, T-24 bladder cancer, C-33A cervical carcinoma, HT-3 metastatic cervical carcinoma, SiHa squamous cervical carcinoma, CaSki epidermoid cervical carcinoma, NCI-H292 mucoepidermoid lung carcinoma, NCI-2030, non small cell lung carcinoma, HeLa, epithelial cervical adenocarcinoma, KB epithelial mouth carcinoma, HT1080 epithelial fibrosarcoma, Saos-2 epithelial osteogenic sarcoma, PC3 epithelial prostate adenocarcinoma, SW480 colorectal carcinoma, CCL-228, MS-751 epidermoid cervical carcinoma, LOX IMVI melanoma, MALME-3M melanoma, M14 melanoma, SK-MEL-2 melanoma, SK-MEL-28 melanoma, SK-MEL-5 melanoma, UACC-257 melanoma, or UACC-62 melanoma cell lines. The specificity can be tested by using cells such as NHLF lung fibroblasts, NHDF dermal fibroblasts, HMEC mammary epithelial cells, PrEC prostate epithelial cells, HRE renal epithelial cells, NHBE bronchial epithelial cells, CoSmC Colon smooth muscle cells, CoEC colon endothelial cells, NHEK epidermal keratinocytes, and bone marrow cells as control cells.

All publications and patents cited in this specification are herein incorporated by reference as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method for treating a patient who has a neoplasm, or inhibiting the development of a neoplasm in a patient who is at risk for developing a neoplasm, said method comprising administering to said patient:
a) a compound having the formula (I): 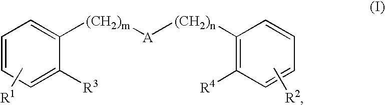
or a pharmaceutically acceptable salt thereof,
wherein A is 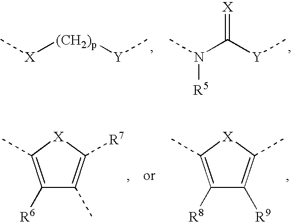
wherein
each of X and Y is, independently, O, NR¹⁰, or S,
each of R⁵ and R¹⁰ is, independently, H or C₁-C₆ alkyl,
each of R⁶, R⁷, R⁸, and R⁹ is, independently, H, C₁-C₆ alkyl, halogen, C₁-C₆
alkyloxy, C₆-C₁₈ aryloxy, or C₆-C₁₈ aryl-C₁-C₆ alkyloxy,
p is an integer between 2 and 6, inclusive,
each of m and n is, independently, an integer between 0 and 2, inclusive,
each of R¹ and R² is 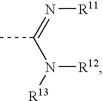
wherein R¹² is H, C₁-C₆ alkyl, C₁-C₈ cycloalkyl, C₁-C₆ alkyloxy-C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent 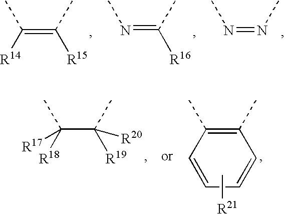
wherein each of R¹⁴, R¹⁵, and R¹⁶ is, independently, H, C₁-C₆ alkyl, halogen, or trifluoromethyl, each of R¹⁷, R¹⁸, R¹⁹, and R²⁰ is, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl,
each of R³ and R⁴ is, independently, H, Cl, Br, OH, OCH₃, OCF₃, NO₂, and NH₂, or R³ and R⁴ together form a single bond; and
b) one or more Group A antiproliferative agent(s),
wherein said compound of formula (I) and said Group A antiproliferative agent(s) are administered simultaneously, or within 14 days of each other, in amounts sufficient to inhibit the growth of said neoplasm.

2. The method of claim 1, wherein said Group A antiproliferative agent is vinblastine, carboplatin, etoposide, or gemcitabine.

3. The method of claim 1, wherein said compound of formula (I) is pentamidine, propamidine, butamidine, heptamidine, nonamidine, dibrompropamidine, 2,5-bis(4-amidinophenyl)furan, 2,5-bis(4-amidinophenyl)furan-bis-O-methylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl) thiophene-bis-O-methylamidoxime, 2,4-bis(4-amidinophenyl)thiophene, or 2,4-bis(4-amidinophenyl)thiophene-bis-O-methylamidoxime.

4. The method of claim 1, wherein said compound of formula (I) and said Group A antiproliferative agent(s) are administered within ten days of each other.

5. The method of claim 4, wherein said compound of formula (I) and said Group A antiproliferative agent(s) are administered within five days of each other.

6. The method of claim 5, wherein said compound of formula (I) and said Group A antiproliferative agent(s) are administered within twenty-four hours of each other.

7. The method of claim 1, wherein said neoplasm is cancer.

8. The method of claim 7, wherein said cancer is lung cancer.

9. The method of claim 7, wherein said cancer is colon cancer.

10. The method of claim 7, wherein said cancer is a cancer of the ovary.

11. The method of claim 7, wherein said cancer is prostate cancer.

12. The method of claim 7, wherein said cancer is selected from the group consisting of acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma.

13. The method of claim 1, wherein said compound of formula (I) and said Group A antiproliferative agent(s) are each administered to said patient by intravenous, intramuscular, inhalation, rectal, or oral administration.

14. A method for treating a patient who has a neoplasm, or inhibiting the development of a neoplasm in a patient who is at risk for developing a neoplasm, said method comprising administering to said patient:
a) a compound having the formula (I) 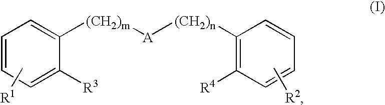
or a pharmaceutically acceptable salt thereof,
wherein A is 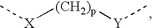
each of X and Y is, independently, O or NH,
p is an integer between 2 and 6, inclusive,
each of m and n is, independently, an integer between 0 and 2, inclusive, wherein the sum of m and n is greater than 0,
each of R¹ and R² is, independently, selected from the group represented by 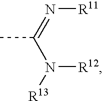
wherein R¹² is H; C₁-C₆ alkyl, C₁-C₈ cycloalkyl, C₁-C₆ alkyloxy C₁-C₆ alkyl, hydroxy C₁-C₆ alkyl, C₁-C₆ alkylamino C₁-C₆ alkyl, amino C₁-C₆ alkyl, or, R¹³ is H, C₁-C₆ alkyl, C₁-C₈ cycloalkyl, C₆-C₁₈ aryloxy C₁-C₆ alkyl, C₁-C₆ alkoxy C₁-C₆ alkyl, hydroxy C₁-C₆ alkyl, C₁-C₆ alkylamino C₁-C₆ alkyl, amino C₁-C₆ alkyl, carbo(C₁-C₆ alkoxy), carbo(C₆-C₁₈ aryl-C₁-C₆ alkoxy), carbo(C₆-C₁₈ aryloxy), or C₆-C₁₈ aryl, and R¹¹ is H, OH, or oxy(C₁-C₆ alkyl), or R¹¹ and R¹² together represent 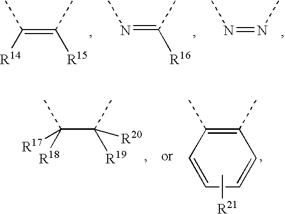
wherein each of R¹⁴, R¹⁵, and R¹⁶ is, independently, H, C₁-C₆ alkyl, halogen, or trifluoromethyl, each of R¹⁷, R¹⁸, R¹⁹, and R²⁰ are, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl,
each of R³ and R⁴ is, independently, H, Cl, Br, OH, OCH₃, OCF₃, NO₂, and NH₂, or R³ and R⁴ together form a single bond;
or A is 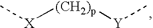
each of X and Y is, independently, O or NH,
p is an integer between 2 and 6, inclusive,
each of m and n is 0, and
each of R¹ and R² is, independently, selected from the group represented by 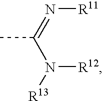
wherein R¹² is C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent 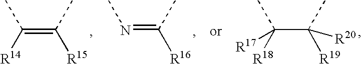
wherein each of R¹⁴, R¹⁵, and R¹⁶ is, independently, H, C₁-C₆ alkyl, halogen, or trifluoromethyl, each of R¹⁷, R¹⁸, and R¹⁹ is, independently, H or C₁-C₆ alkyl, and R²⁰ is C₁-C₆ alkyl, C₁-C₆ alkyloxy, or trifluoromethyl;
or A is 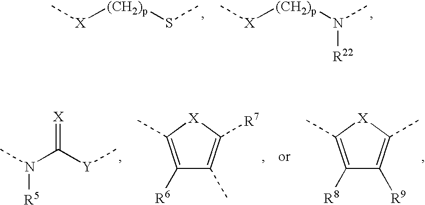
each of X and Y is, independently, O, NR¹⁰, or S,
each of R⁵ and R¹⁰ is, independently, H or C₁-C₆ alkyl,
each of R⁶, R⁷, R⁸, and R⁹ is, independently, H, C₁-C₆ alkyl, halogen, C₁-C₆
alkyloxy, C₆-C₁₈ aryloxy, or C₆-C₁₈ aryl C₁-C₆ alkyloxy,
R²² is C₁-C₆ alkyl,
p is an integer between 2 and 6, inclusive,
each of m and n is, independently, an integer between 0 and 2, inclusive,
each of R¹ and R² is, independently, selected from the group represented by 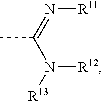
wherein R¹² is H, C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₆-C₁₈ aryloxy C₁-C₆ alkyl, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent 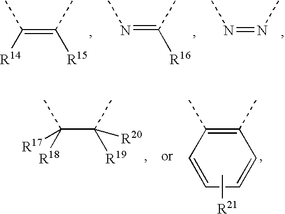
wherein each of R¹⁴, R¹⁵, and R¹⁶ is, independently, H, C₁-C₆ alkyl, halogen, or trifluoromethyl, each of R¹⁷, R¹⁸, R¹⁹, and R²⁰ are, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, or C₆-C₁₈ aryl, and
each of R³ and R⁴ is, independently, H, Cl, Br, OH, OCH₃, OCF₃, NO₂, and NH₂, or R³ and R⁴ together form a single bond, and
b) one or more Group A and/or Group B antiproliferative agent(s),
wherein said compound of formula (I) and said Group A and/or Group B antiproliferative agent(s) are administered simultaneously, or within 14 days of each other, in amounts sufficient to inhibit the growth of said neoplasm.

15. The method of claim 14, wherein said Group A and/or Group B antiproliferative agent is vinblastine, carboplatin, adriamycin (doxorubicin), etoposide, or gemcitabine.

16. The method of claim 14, wherein said compound of formula (I) and said Group A and/or Group B antiproliferative agent(s) are administered within ten days of each other.

17. The method of claim 16, wherein said compound of formula (I) and said Group A and/or Group B antiproliferative agent(s) are administered within five days of each other.

18. The method of claim 17, wherein said compound of formula (I) and said Group A and/or Group B antiproliferative agent(s) are administered within twenty-four hours of each other.

19. The method of claim 14, wherein said neoplasm is cancer.

20. The method of claim 19, wherein said cancer is lung cancer.

21. The method of claim 19, wherein said cancer is colon cancer.

22. The method of claim 19, wherein said cancer is a cancer of the ovary.

23. The method of claim 19, wherein said cancer is prostate cancer.

24. The method of claim 19, wherein said cancer is selected from the group consisting of acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma.

25. The method of claim 14, wherein said compound of formula (I) and said Group A and/or Group B antiproliferative agents are each administered to said patient by intravenous, intramuscular, inhalation, rectal, or oral administration.

26. A method for treating a patient who has a neoplasm, or inhibiting the development of a neoplasm in a patient who is at risk for developing a neoplasm, said method comprising administering to said patient:
a) a compound selected from propamidine, butamidine, heptamidine, nonamidine, stilbamidine, hydroxystilbamidine, diminazene, benzamidine, phenamidine, dibrompropamidine, 1,3-bis(4-amidino-2-methoxyphenoxy)propane, netropsin, distamycin, phenamidine, amicarbalide, bleomycin, actinomycin, daunorubicin, 1,3-bis(4-amidino-2-methoxyphenoxy)propane, phenamidine, amicarbalide, 1,5-bis(4′-(N-hydroxyamidino)phenoxy)pentane, 1,3-bis(4′-(N-hydroxyamidino)phenoxy)propane, 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-O-methylamidoxime, 2,5-bis[4-amidinophenyl]furan-bis-O-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl)thiophene, 2,5-bis(4-amidinophenyl)thiophene-bis-O-methylamidoxime, 2,4-bis(4-amidinophenyl)thiophene, 2,4-bis(4-amidinophenyl)thiophene-bis-O-methylamidoxime, 2,8-diamidinodibenzothiophene, 2,8-bis(N-isopropylamidino)carbazole, 2,8-bis(N-hydroxyamidino)carbazole, 2,8-bis(2-imidazolinyl)dibenzothiophene, 2,8-bis(2-imidazolinyl)-5,5-dioxodibenzothiophene, 3,7-diamidinodibenzothiophene, 3,7-bis(N-isopropylamidino)dibenzothiophene, 3,7-bis(N-hydroxyamidino)dibenzothiophene, 3,7-diaminodibenzothiophene, 3,7-dibromodibenzothiophene, 3,7-dicyanodibenzothiophene, 2,8-diamidinodibenzofuran, 2,8-di(2-imidazolinyl)dibenzofuran, 2,8-di(N-isopropylamidino)dibenzofuran, 2,8-di(N-hydroxylamidino)dibenzofuran, 3,7-di(2-imidazolinyl)dibenzofuran, 3,7-di(isopropylamidino)dibenzofuran, 3,7-di(N-hydroxylamidino)dibenzofuran, 2,8-dicyanodibenzofuran, 4,4′-dibromo-2,2′-dinitrobiphenyl, 2-methoxy-2′-nitro-4,4′-dibromobiphenyl, 2-methoxy-2′-amino-4,4′-dibromobiphenyl, 3,7-dibromodibenzofuran, 3,7-dicyanodibenzofuran, 2,5-bis(5-amidino-2-benzimidazolyl)pyrrole, 2,5-bis[5-(2-imidazolinyl)-2-benzimidazolyl]pyrrole, 2,6-bis[5-(2-imidazolinyl)-2-benzimidazolyl]pyridine, 1-methyl-2,5-bis(5-amidino-2-benzimidazolyl)pyrrole, 1-methyl-2,5-bis[5-(2-imidazolyl)-2-benzimidazolyl]pyrrole, 1-methyl-2,5-bis[5-(1,4,5,6-tetrahydro-2-pyrimidinyl)-2-benzimidazolyl]pyrrole, 2,6-bis(5-amidino-2-benzimidazoyl)pyridine, 2,6-bis[5-(1,4,5,6-tetrahydro-2-pyrimidinyl)-2-benzimidazolyl]pyridine, 2,5-bis(5-amidino-2-benzimidazolyl)furan, 2,5-bis-[5-(2-imidazolinyl)-2-benzimidazolyl]furan, 2,5-bis-(5-N-isopropylamidino-2-benzimidazolyl)furan, 2,5-bis-(4-guanylphenyl)furan, 2,5-bis(4-guanylphenyl)-3,4-dimethylfuran, 2,5-bis {p-[2-(3,4,5,6-tetrahydropyrimidyl)phenyl]}furan, 2,5-bis[4-(2-imidazolinyl)phenyl]furan, 2,5 [bis-{4-(2-tetrahydropyrimidinyl)}phenyl]-3-(p-tolyloxy)furan, 2,5 [bis {4-(2-imidazolinyl)}phenyl]-3-(p-tolyloxy)furan, 2,5-bis {4-[5-(N-2-aminoethylamido)benzimidazol-2-yl]phenyl}furan, 2,5-bis[4-(3 a,4,5,6,7,7a-hexahydro-1H-benzimidazol-2-yl)phenyl]furan, 2,5-bis[4-(4,5,6,7-tetrahydro-1H-1,3-diazepin-2-yl)phenyl]furan, 2,5-bis(4-N,N-dimethylcarboxhydrazidephenyl)furan, 2,5-bis {4-[2-(N-2-hydroxyethyl)imidazolinyl]phenyl}furan, 2,5-bis[4-(N-isopropylamidino)phenyl]furan, 2,5-bis{4-[3-(dimethylaminopropyl)amidino]phenyl} furan, 2,5-bis {4-[N-(3-aminopropyl)amidino]phenyl}furan, 2,5-bis[2-(imidzaolinyl)phenyl]-3,4-bis(methoxymethyl)furan, 2,5-bis[4-N-(dimethylaminoethyl)guanyl]phenylfuran, 2,5-bis{4-[(N-2-hydroxyethyl)guanyl]phenyl}furan, 2,5-bis[4-N-(cyclopropylguanyl) phenyl]furan, 2,5-bis[4-(N,N-diethylaminopropyl)guanyl]phenylfuran, 2,5-bis{4-[2-(N-ethylimidazolinyl)]phenyl}furan, 2,5-bis {4-[N-(3-pentylguanyl)]}phenylfuran, 2,5-bis[4-(2-imidazolinyl)phenyl]-3-methoxyfuran, 2,5-bis[4-(N-isopropylamidino) phenyl]-3-methylfuran, bis[5-amidino-2-benzimidazolyl]methane, bis[5-(2-imidazolyl)-2-benzimidazolyl]methane, 1,2-bis[5-amidino-2-benzimidazolyl]ethane, 1,2-bis[5-(2-imidazolyl)-2-benzimidazolyl]ethane, 1,3-bis[5-amidino-2-benzimidazolyl]propane, 1,3-bis[5-(2-imidazolyl)-2-benzimidazolyl]propane, 1,4-bis[5-amidino-2-benzimidazolyl]propane, 1,4-bis[5-(2-imidazolyl)-2-benzimidazolyl]butane, 1,8-bis[5-amidino-2-benzimidazolyl]octane, trans-1,2-bis[5-amidino-2-benzimidazolyl]ethene, 1,4-bis[5-(2-imidazolyl)-2-benzimidazolyl]-1-butene, 1,4-bis[5-(2-imidazolyl)-2-benzimidazolyl]-2-butene, 1,4-bis[5-(2-imidazolyl)-2-benzimidazolyl]-1-methylbutane, 1,4-bis[5-(2-imidazolyl)-2-benzimidazolyl]-2-ethylbutane, 1,4-bis[5-(2-imidazolyl)-2-benzimidazolyl]-1-methyl-1-butene, 1,4-bis[5-(2-imidazolyl)-2-benzimidazolyl]-2,3-diethyl-2-butene, 1,4-bis[5-(2-imidazolyl)-2-benzimidazolyl]-1,3-butadiene, 1,4-bis[5-(2-imidazolyl)-2-benzimidazolyl]-2-methyl-1,3-butadiene, bis[5-(2-pyrimidyl)-2-benzimidazolyl]methane, 1,2-bis[5-(2-pyrimidyl)-2-benzimidazolyl]ethane, 1,3-bis[5-amidino-2-benzimidazolyl]propane, 1,3-bis[5-(2-pyrimidyl)-2-benzimidazolyl]propane, 1,4-bis[5-(2-pyrimidyl)-2-benzimidazolyl]butane, 1,4-bis[5-(2-pyrimidyl)-2-benzimidazolyl]-1-butene, 1,4-bis[5-(2-pyrimidyl)-2-benzimidazolyl]-2-butene, 1,4-bis[5-(2-pyrimidyl)-2-benzimidazolyl]-1-methylbutane, 1,4-bis[5-(2-pyrimidyl)-2-benzimidazolyl]-2-ethylbutane, 1,4-bis[5-(2-pyrimidyl)-2-benzimidazolyl]-1-methyl-1-butene, 1,4-bis[5-(2-pyrimidyl)-2-benzimidazolyl]-2,3-diethyl-2-butene, 1,4-bis[5-(2-pyrimidyl)-2-benzimidazolyl]-1,3-butadiene, and 1,4-bis[5-(2-pyrimidyl)-2-benzimidazolyl]-2-methyl-1,3-butadiene, 2,4-bis(4-guanylphenyl)pyrimidine, 2,4-bis(4-imidazolin-2-yl)pyrimidine, 2,4-bis[(tetrahydropyrimidinyl-2-yl)phenyl]pyrimidine, 2-(4-[N-1-propylguanyl]phenyl)-4-(2-methoxy-4-[N-1-propylguanyl]phenyl)pyrimidine, 4-(N-cyclopentylamidino)-1,2-phenylene diamine, 2,5-bis-[2-(5-amidino)benzimidazoyl]furan, 2,5-bis[2-{5-(2-imidazolino)}benzimidazoyl]furan, 2,5-bis[2-(5-N-isopropylamidino)benzimidazoyl]furan, 2,5-bis[2-(5-N-cyclopentylamidino)benzimidazoyl]furan, 2,5-bis[2-(5-amidino)benzimidazoyl]pyrrole, 2,5-bis[2-{5-(2-imidazolino)}benzimidazoyl]pyrrole, 2,5-bis[2-(5-N-isopropylamidino)benzimidazoyl]pyrrole, 2,5-bis[2-(5-N-cyclopentylamidino)benzimidazoyl]pyrrole, 1-methyl-2,5-bis[2-(5-amidino)benzimidazoyl]pyrrole, 2,5-bis[2-{5-(2-imidazolino)}benzimidazoyl]-1-methylpyrrole, 2,5-bis[2-(5-N-cyclopentylamidino)benzimidazoyl]-1-methylpyrrole, 2,5-bis[2-(5-N-isopropylamidino)benzimidazoyl]thiophene, 2,6-bis[2-{5-(2-imidazolino)}benzimidazoyl]pyridine, 2,6-bis[2-(5-amidino)benzimidazoyl]pyridine, 4,4′-bis[2-(5-N-isopropylamidino)benzimidazoyl]-1,2-diphenylethane, 4,4′-bis[2-(5-N-cyclopentylamidino)benzimidazoyl]-2,5-diphenylfuran, 2,5-bis[2-(5-amidino)benzimidazoyl]benzo[b]furan, 2,5-bis[2-(5-N-cyclopentylamidino)benzimidazoyl]benzo[b]furan, 2,7-bis[2-(5-N-isopropylamidino)benzimidazoyl]fluorene, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N,N⁸,N¹¹-trimethylaminopropylcarbamoyl)phenyl]furan, 2,5-bis[3-amidinophenyl]furan, 2,5-bis [3-(N-isopropylamidino)amidinophenyl]furan, 2,5-bis[3[(N-(2-dimethylaminoethyl)amidino]phenylfuran, 2,5-bis[4-(N-2,2,2-trichloroethoxycarbonyl)amidinophenyl]furan, 2,5-bis[4-(N-thioethylcarbonyl) amidinophenyl]furan, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis[4-(N-(4-methoxy)phenoxycarbonyl)amidinophenyl]furan, 2,5-bis[4(1-acetoxyethoxycarbonyl)amidinophenyl]furan, and 2,5-bis[4-(N-(3-fluoro)phenoxycarbonyl)amidinophenyl]furan, or a pharmaceutically acceptable salt thereof, and
b) one or more Group A and/or one or more Group B antiproliferative agent(s),
wherein said compound and said Group A and/or one or more Group B antiproliferative agent(s) are administered simultaneously or within 14 days of each other, in amounts sufficient to treat or inhibit the development of a neoplasm in said patient.

27. The method of claim 26 wherein said Group A or Group B antiproliferative agent is vinblastine, carboplatin, adriamycin (doxorubicin), etoposide, or gemcitabine.

28. The method of claim 26, wherein said compound of formula (I) and Group A or Group B antiproliferative agent(s) are administered within ten days of each other.

29. The method of claim 28, wherein said compound of formula (I) and said Group A or Group B antiproliferative agent(s) are administered within five days of each other.

30. The method of claim 29, wherein said compound of formula (I) and said Group A or Group B antiproliferative agent(s) are administered within twenty-four hours of each other.

31. The method of claim 26, wherein said neoplasm is cancer.

32. The method of claim 31, wherein said cancer is lung cancer.

33. The method of claim 31, wherein said cancer is colon cancer.

34. The method of claim 31, wherein said cancer is a cancer of the ovary.

35. The method of claim 31, wherein said cancer is prostate cancer.

36. The method of claim 31, wherein said cancer is selected from the group consisting of acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma.

37. The method of claim 26, wherein said compound of formula (I) and said Group A or Group B antiproliferative agent(s) are administered to said patient by intravenous, intramuscular, inhalation, rectal, or oral administration.

38. A method for treating a patient who has a neoplasm, or inhibiting the development of a neoplasm in a patient who is at risk for developing a neoplasm, said method comprising administering to said patient:
a) an endo-exonuclease inhibitor; and
b) one or more Group A antiproliferative agent(s), wherein said endo-exonuclease inhibitor and said Group A antiproliferative agents are administered simultaneously, or within 14 days of each other, in amounts sufficient to inhibit the growth of said neoplasm.

39. The method of claim 38, wherein said Group A antiproliferative agent(s) are selected from vinblastine, carboplatin, etoposide, or gemcitabine.

40. The method of claim 38, wherein said endo-exonuclease inhibitor and said Group A antiproliferative agent(s) are administered within ten days of each other.

41. The method of claim 40, wherein said endo-exonuclease inhibitor and said Group A antiproliferative agent(s) are administered within five days of each other.

42. The method of claim 41, wherein said endo-exonuclease inhibitor and said Group A antiproliferative agent(s) are administered within twenty-four hours of each other.

43. The method of claim 38, wherein said neoplasm is cancer.

44. The method of claim 43, wherein said cancer is lung cancer.

45. The method of claim 43, wherein said cancer is colon cancer.

46. The method of claim 43, wherein said cancer is a cancer of the ovary.

47. The method of claim 43, wherein said cancer is prostate cancer.

48. The method of claim 43, wherein said cancer is selected from the group consisting of acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma.

49. The method of claim 38, wherein said endo-exonuclease inhibitor and said Group A antiproliferative agent(s) are each administered to said patient by intravenous, intramuscular, inhalation, rectal, or oral administration.

50. A method for treating a patient who has a neoplasm, or inhibiting the development of a neoplasm in a patient who is at risk for developing a neoplasm, said method comprising administering to said patient:
a) a PRL phosphatase inhibitor or a PTP1B inhibitor; and
b) one or more Group A antiproliferative agent(s), wherein said endo-exonuclease inhibitor and said antiproliferative agents are administered simultaneously, or within 14 days of each other, in amounts sufficient to inhibit the growth of said neoplasm.

51. The method of claim 50, wherein said Group A antiproliferative agent(s) is selected from vinblastine, carboplatin, etoposide, or gemcitabine.

52. The method of claim 50, wherein said inhibitor and said Group A antiproliferative agent(s) are administered within ten days of each other.

53. The method of claim 52, wherein said inhibitor and said Group A antiproliferative agent(s) are administered within five days of each other.

54. The method of claim 53, wherein said inhibitor and said Group A antiproliferative agent(s) are administered within twenty-four hours of each other.

55. The method of claim 50, wherein said neoplasm is cancer.

56. The method of claim 55, wherein said cancer is lung cancer.

57. The method of claim 55, wherein said cancer is colon cancer.

58. The method of claim 55, wherein said cancer is a cancer of the ovary.

59. The method of claim 55, wherein said cancer is prostate cancer.

60. The method of claim 55, wherein said cancer is selected from the group consisting of acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendriglioma, schwannoma, meningioma, melanoma, neuroblastoma, and retinoblastoma.

61. The method of claim 50, wherein said inhibitor and said Group A antiproliferative agent(s) are administered to said patient by intravenous, intramuscular, inhalation, rectal, or oral administration.

62. A method for treating a neoplastic cell, said method comprising contacting said cell with:
a) a compound having the formula (I): 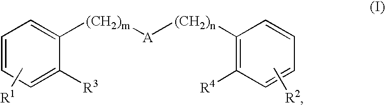
or a pharmaceutically acceptable salt thereof,
wherein A is 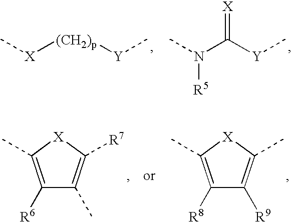
wherein
each of X and Y is, independently, O, NR¹⁰, or S,
each of R⁵ and R¹⁰ is, independently, H or C₁-C₆ alkyl,
each of R⁶, R⁷, R⁸, and R⁹ is, independently, H, C₁-C₆ alkyl, halogen, C₁-C₆
alkyloxy, C₆-C₁₈ aryloxy, or C₆-C₁₈ aryl-C₁-C₆ alkyloxy,
p is an integer between 2 and 6, inclusive,
each of m and n is, independently, an integer between 0 and 2, inclusive,
each of R¹ and R² is 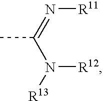
wherein R¹² is H, C₁-C₆ alkyl, C₁-C₈ cycloalkyl, C₁-C₆ alkyloxy-C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent 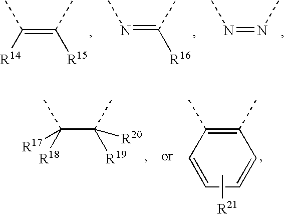
wherein each of R¹⁴, R¹⁵, and R¹⁶ is, independently, H, C₁-C₆ alkyl, halogen, or trifluoromethyl, each of R¹⁷, R¹⁸, R¹⁹, and R²⁰ is, independently, H or C₁-C₆alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl,
each of R³ and R⁴ is, independently, H, Cl, Br, OH, OCH₃, OCF₃, NO₂, and NH₂, or R³ and R⁴ together form a single bond; and
b) one or more Group A antiproliferative agent(s),
wherein said compound of formula (I) and said Group A antiproliferative agent(s) are administered simultaneously, or within 14 days of each other, in amounts sufficient to inhibit the growth of said neoplastic cell.

63. The method of claim 62, wherein said Group A antiproliferative agent is vinblastine, carboplatin, etoposide, or gemcitabine.

64. A method for treating a neoplastic cell, said method comprising contacting said cell with:
a) a compound having the formula (I) 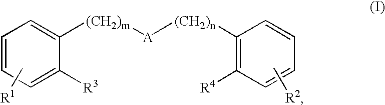
or a pharmaceutically acceptable salt thereof, wherein A is 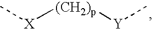
each of X and Y is, independently, O or NH,
p is an integer between 2 and 6, inclusive,
each of m and n is, independently, an integer between 0 and 2, inclusive, wherein the sum of m and n is greater than 0,
each of R¹ and R² is, independently, selected from the group represented by 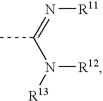
wherein R¹² is H, C₁-C₆ alkyl, C₁-C₈ cycloalkyl, C₁-C₆ alkyloxy C₁-C₆ alkyl, hydroxy C₁-C₆ alkyl, C₁-C₆ alkylamino C₁-C₆ alkyl, amino C₁-C₆ alkyl, or, R¹³ is H, C₁-C₆ alkyl, C₁-C₈ cycloalkyl, C₆-C₁₈ aryloxy C₁-C₆ alkyl, C₁-C₆ alkoxy C₁-C₆ alkyl, hydroxy C₁-C₆ alkyl, C₁-C₆ alkylamino C₁-C₆ alkyl, amino C₁-C₆ alkyl, carbo(C₁-C₆ alkoxy), carbo(C₆-C₁₈ aryl-C₁-C₆ alkoxy), carbo(C₆-C₁₈ aryloxy), or C₆-C₁₈ aryl, and R¹¹ is H, OH, or oxy(C₁-C₆ alkyl), or R¹¹ and R¹² together represent 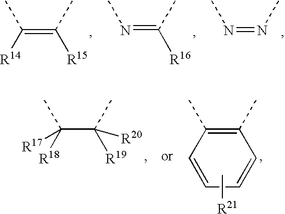
wherein each of R¹⁴, R¹⁵, and R¹⁶ is, independently, H, C₁-C₆ alkyl, halogen, or trifluoromethyl, each of R¹⁷, R¹⁸, R¹⁹, and R²⁰ are, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl,
each of R³ and R⁴ is, independently, H, Cl, Br, OH, OCH₃, OCF₃, NO₂, and NH₂, or R³ and R⁴ together form a single bond;
or A is 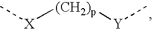
each of X and Y is, independently, O or NH,
p is an integer between 2 and 6, inclusive,
each of m and n is 0, and
each of R¹ and R² is, independently, selected from the group represented by 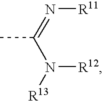
wherein R¹² is C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent 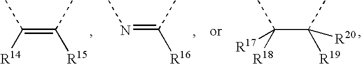
wherein each of R¹⁴, R¹⁵, and R¹⁶ is, independently, H, C₁-C₆ alkyl, halogen, or trifluoromethyl, each of R¹⁷, R¹⁸, and R¹⁹ is, independently, H or C₁-C₆ alkyl, and R²⁰ is C₁-C₆ alkyl, C₁-C₆ alkyloxy, or trifluoromethyl;
or A is 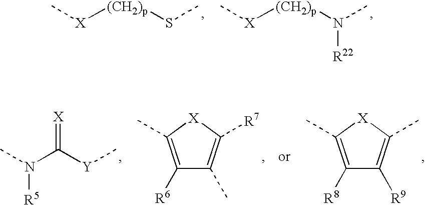
each of X and Y is, independently, O, NR¹⁰, or S,
each of R⁵ and R¹⁰ is, independently, H or C₁-C₆ alkyl,
each of R⁶, R⁷, R⁸, and R⁹ is, independently, H, C₁-C₆ alkyl, halogen, C₁-C₆
alkyloxy, C₆-C₁₈ aryloxy, or C₆-C₁₈ aryl C₁-C₆ alkyloxy,
R²² is C₁-C₆ alkyl,
p is an integer between 2 and 6, inclusive,
each of m and n is, independently, an integer between 0 and 2, inclusive,
each of R¹ and R² is, independently, selected from the group represented by 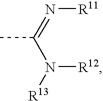
wherein R¹² is H, C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆alkyl, or C₆-C₁₈ aryl, R¹³ is H, C₁-C₆ alkyl, C₁-C₈ cycloalkyl, C₆-C₁₈ aryloxy C₁-C₆ alkyl, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent 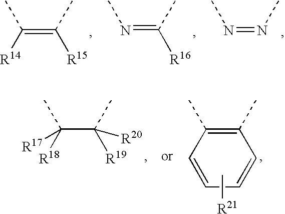
wherein each of R¹⁴, R¹⁵, and R¹⁶ is, independently, H, C₁-C₆ alkyl, halogen, or trifluoromethyl, each of R¹⁷, R¹⁸, R¹⁹, and R²⁰ are, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, or C₆-C₁₈ aryl, and
each of R³ and R⁴ is, independently, H, Cl, Br, OH, OCH₃, OCF₃, NO₂, and NH₂, or R³ and R⁴ together form a single bond, and
b) one or more Group A and/or Group B antiproliferative agent(s),
wherein said compound of formula (I) and said Group A and/or Group B antiproliferative agent(s) are administered simultaneously, or within 14 days of each other, in amounts sufficient to inhibit the growth of said neoplastic cell.

65. The method of claim 64, wherein said Group A and/or Group B antiproliferative agent is vinblastine, carboplatin, adriamycin (doxorubicin), etoposide, or gemcitabine.

66. A method for treating a neoplastic cell, said method comprising contacting said cell with:
a) an endo-exonuclease inhibitor; and
b) one or more Group A antiproliferative agents, wherein said endo-exonuclease inhibitor and said Group A antiproliferative agents are administered simultaneously, or within 14 days of each other, in amounts sufficient to inhibit the growth of said neoplastic cell.

67. The method of claim 66, wherein said Group A antiproliferative agents are selected from vinblastine, carboplatin, etoposide, or gemcitabine.

68. A method for treating a neoplastic cell, said method comprising contacting said cells with:
a) a PRL phosphatase inhibitor or a PTP1B inhibitor; and
b) one or more Group A antiproliferative agent(s), wherein said endo-exonuclease inhibitor and said antiproliferative agents are administered simultaneously, or within 14 days of each other, in amounts sufficient to inhibit the growth of said neoplastic cell.

69. The method of claim 68, wherein said Group A antiproliferative agent is selected from vinblastine, carboplatin, etoposide, or gemcitabine.

70. A composition comprising:
a) a compound having the formula (I): 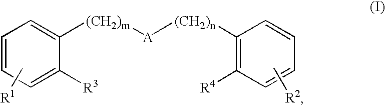
or a pharmaceutically acceptable salt thereof,
wherein A is 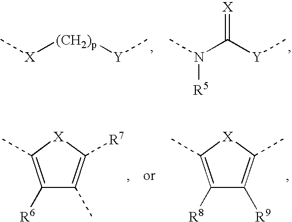
wherein
each of X and Y is, independently, O, NR¹⁰, or S,
each of R⁵ and R¹⁰ is, independently, H or C₁-C₆ alkyl,
each of R⁶, R⁷, R⁸, and R⁹ is, independently, H, C₁-C₆ alkyl, halogen, C₁-C₆
alkyloxy, C₆-C₁₈ aryloxy, or C₆-C₁₈ aryl-C₁-C₆ alkyloxy,
p is an integer between 2 and 6, inclusive,
each of m and n is, independently, an integer between 0 and 2, inclusive,
each of R¹ and R² is 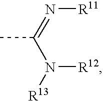
wherein R¹² is H, C₁-C₆ alkyl, C₁-C₈ cycloalkyl, C₁-C₆ alkyloxy-C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent 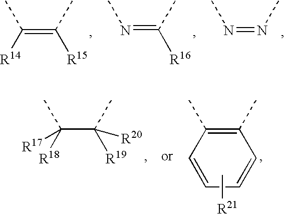
wherein each of R¹⁴, R¹⁵, and R¹⁶ is, independently, H, C₁-C₆ alkyl, halogen, or trifluoromethyl, each of R¹⁷, R¹⁸, R¹⁹, and R²⁰ is, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl,
each of R³ and R⁴ is, independently, H, Cl, Br, OH, OCH₃, OCF₃, NO₂, and NH₂, or R³ and R⁴ together form a single bond; and
b) one or more Group A antiproliferative agent(s).

71. The composition of claim 70, wherein said Group A antiproliferative agent is vinblastine, carboplatin, etoposide, or gemcitabine.

72. A composition comprising:
a) a compound having the formula (I) 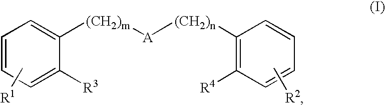
or a pharmaceutically acceptable salt thereof,
wherein A is 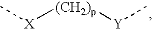
each of X and Y is, independently, O or NH,
p is an integer between 2 and 6, inclusive,
each of m and n is, independently, an integer between 0 and 2, inclusive, wherein the sum of m and n is greater than 0,
each of R¹ and R² is, independently, selected from the group represented by 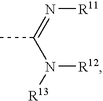
wherein R¹² is H, C₁-C₆ alkyl, C₁-C₈ cycloalkyl, C₁-C₆ alkyloxy C₁-C₆ alkyl, hydroxy C₁-C₆ alkyl, C₁-C₆ alkylamino C₁-C₆ alkyl, amino C₁-C₆ alkyl, or, R¹³ is H, C₁-C₆ alkyl, C₁-C₈ cycloalkyl, C₆-C₁₈ aryloxy C₁-C₆ alkyl, C₁-C₆ alkoxy C₁-C₆ alkyl, hydroxy C₁-C₆ alkyl, C₁-C₆ alkylamino C₁-C₆ alkyl, amino C₁-C₆ alkyl, carbo(C₁-C₆ alkoxy), carbo(C₆-C₁₈ aryl-C₁-C₆ alkoxy), carbo(C₆-C₁₈ aryloxy), or C₆-C₁₈ aryl, and R¹¹ is H, OH, or oxy(C₁-C₆ alkyl), or R¹¹ and R¹² together represent 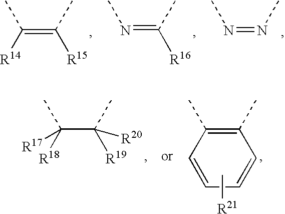
wherein each of R¹⁴, R¹⁵, and R¹⁶ is, independently, H, C₁-C₆ alkyl, halogen, or trifluoromethyl, each of R¹⁷, R¹⁸, R¹⁹, and R²⁰ are, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl,
each of R³ and R⁴ is, independently, H, Cl, Br, OH, OCH₃, OCF₃, NO₂, and NH₂, or R³ and R⁴ together form a single bond;
or A is 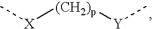
each of X and Y is, independently, O or NH,
p is an integer between 2 and 6, inclusive,
each of m and n is 0, and
each of R¹ and R² is, independently, selected from the group represented by 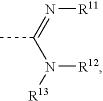
wherein R¹² is C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent 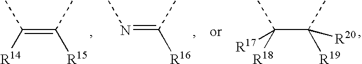
wherein each of R¹⁴, R¹⁵, and R¹⁶ is, independently, H, C₁-C₆ alkyl, halogen, or trifluoromethyl, each of R¹⁷, R¹⁸, and R¹⁹ is, independently, H or C₁-C₆ alkyl, and R²⁰ is C₁-C₆ alkyl, C₁-C₆ alkyloxy, or trifluoromethyl;
or A is 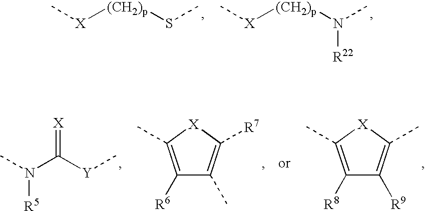
each of X and Y is, independently, O, NR¹⁰, or S,
each of R⁵ and R¹⁰ is, independently, H or C₁-C₆ alkyl,
each of R⁶, R⁷, R⁸, and R⁹ is, independently, H, C₁-C₆ alkyl, halogen, C₁-C₆
alkyloxy, C₆-C₁₈ aryloxy, or C₆-C₁₈ aryl C₁-C₆ alkyloxy,
R²² is C₁-C₆ alkyl,
p is an integer between 2 and 6, inclusive,
each of m and n is, independently, an integer between 0 and 2, inclusive,
each of R¹ and R² is, independently, selected from the group represented by 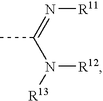
wherein R¹² is H, C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₆-C₁₈ aryloxy C₁-C₆ alkyl, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent 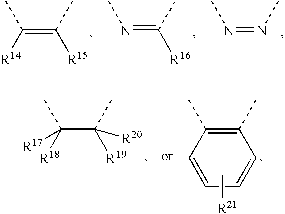
wherein each of R¹⁴, R¹⁵, and R¹⁶ is, independently, H, C₁-C₆ alkyl, halogen, or trifluoromethyl, each of R¹⁷, R¹⁸, R¹⁹, and R²⁰ are, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, or C₆-C₁₈ aryl, and
each of R³ and R⁴ is, independently, H, Cl, Br, OH, OCH₃, OCF₃, NO₂, and NH₂, or R³ and R⁴ together form a single bond, and
b) one or more Group A and/or Group B antiproliferative agent(s).

73. The composition of claim 72, wherein said Group A and/or Group B antiproliferative agent is vinblastine, carboplatin, adriamycin (doxorubicin), etoposide, or gemcitabine.
